(19) United States
(12) Patent Application Publication
(10) Pub. No.: US 2024/0228607 A9
(43) Pub. Date: Jul. 11, 2024

(54) METHODS FOR TREATING HIGH RISK MYELODYSPLASTIC SYNDROMES

(71) Applicant: Northwestern University, Evanston, IL (US)

(72) Inventors: Peng Ji, Evanston, IL (US); Kehan Ren, Evanston, IL (US); Yang Mei, Evanston, IL (US)

(21) Appl. No.: 18/347,541

(22) Filed: Jul. 5, 2023

Related U.S. Application Data
(60) Provisional application No. 63/367,912, filed on Jul. 7, 2022.

(51) Int. Cl. C07K 16/24; A61P 35/02; C07K 14/715; C07K 16/28; G01N 33/50

(52) U.S. Cl. CPC C07K 16/248; A61P 35/02; C07K 14/715; C07K 16/2866; G01N 33/5011; G01N 2800/7028

(57) ABSTRACT

The present invention provides methods of using an inhibitor of the IL-6 signaling pathway to inhibit disease progression in a subject with a high-risk myelodysplastic syndrome (MDS) or treat low blast count acute myeloid leukemia (AML). Methods for identifying therapeutics for preventing MDS to AML progression are also provided.

### CROSS-REFERENCE TO RELATED APPLICATIONS

This application claims priority to U.S. Provisional Application No. 63/367,912 filed on Jul. 7, 2022, the content of which is incorporated by reference in its entirety.

### STATEMENT REGARDING FEDERALLY SPONSORED RESEARCH

This invention was made with government support under grant numbers W81XWH1910228P00001 and W81XWH1910228 awarded by the Department of Defense, Department of the Army, U.S. Army Medical Research and Materiel Command. The government has certain rights in this invention.

### BACKGROUND

Approximately 20,000 people in the United States are diagnosed with myelodysplastic syndromes (MDS) each year. MDS is predominantly a disease of the elderly with a median age of diagnosis of 71. MDS are a group of cancers that keep your blood stem cells from maturing into healthy blood cells. Patients with high risk MDS have a significantly increased risk of progression into acute myeloid leukemia (AML).

Allogeneic stem cell transplantation (ASCT) remains the only curative treatment option for high risk MDS. Prolonged disease-free survival occurs in 35%-50% of ASCT-treated patients. However, many patients with high risk MDS are ineligible for ASCT or intensive chemotherapy due to age limitations that reflect potential complications. The current standard of care for these ineligible patients is treatment with hypomethylating agents (HMAs), but unfortunately this disease eventually becomes drug-resistant and progresses to secondary acute myeloid leukemia (sAML). Patients with sAML usually have a poor prognosis, with a 4- to 6-month median survival duration.

Accordingly, there remains a need in the art for alternative treatments for high risk MDS that are safe for use by the elderly.

### SUMMARY

In an aspect, the present disclosure provides a method of inhibiting disease progression in a subject with a high-risk myelodysplastic syndrome (MDS), the method comprising administering to the subject an inhibitor of the IL-6 signaling pathway in an amount effective to inhibit disease progression.

In embodiments, the method inhibits the development of acute myeloid leukemia (AML) in the subject compared to an untreated control. In embodiments, the survival of the subject is extended compared to an untreated control. In embodiments, the method reduces the growth of blasts in the subject. In embodiments, the blasts are CD34+ cells. In embodiments, the blasts are CD34 negative cells that are morphologically defined as blasts.

In embodiments, the inhibitor is an anti-IL-6 antibody or an anti-IL-6R antibody. In embodiments, the inhibitor is tocilizumab. In embodiments, the inhibitor is a recombinant gp130 Fc chimera protein. In embodiments, the subject is a human.

In another aspect, provided herein is a method of treating low-blast count acute myeloid leukemia (AML) in a subject, the method comprising administering an inhibitor of the IL-6 signaling pathway in an amount effective to treat the AML. In embodiments, the inhibitor is an anti-IL-6 antibody or an anti-IL-6R antibody. In embodiments, the inhibitor is tocilizumab. In embodiments, the inhibitor is a recombinant gp130 Fc chimera protein. In embodiments, the subject is a human.

In another aspect, provided herein is a method for identifying a therapeutic for inhibiting MDS to AML progression, the method comprising transplanting bone marrow cells from a mDia1/miR-146a double knockout (DKO) animals having MDS into a test animal; administering a candidate therapeutic to the test animal; and measuring the progression of MDS to AML in the test animal, wherein a decrease in the progression of MDS to AML in the test animal compared to an untreated control animal transplanted with bone marrow cells from the DKO animal identifies the therapeutic.

In embodiments, the progression of MDS to AML is measured by survival, wherein increased survival in the test animal compared to the control animal indicates a decrease in progression of MDS to AML. In embodiments, the progression of MDS to AML is measured by the growth of blasts, wherein reduced growth of blasts in the test animal compared to the control animal indicates a decrease in progress of MDS to AML. In embodiments, the progression of MDS to AML is measured by the development of pancytopenia, wherein reduced pancytopenia in the test animal compared to the control animal indicates a decrease in progress of MDS to AML. In embodiments, the test animal is a mouse. In embodiments, the method further comprises lethally irradiating the test animal before the transplanting step. In embodiments, the DKO animal comprises increased levels of IL-6 receptor (IL-6R) in bone marrow compared to a wild-type animal.

### BRIEF DESCRIPTION OF THE DRAWINGS

FIGS. 1A-1E demonstrate that old moribund mDia1/miR-146a DKO mice progress from MDS to acute leukemia. (A) Representative images of H&E staining of the bone marrow and spleens from the indicated mice (12-14-month-old). Scale bars: 100 μm. (B) Representative images of H&E staining showing blasts infiltrating the livers (outlined) of DKO mice from A. Scale bar: 100 μm. (C) Representative images of the bone marrow from moribund DKO mice show osteosclerosis (H&E) and marked fibrosis (reticulin). Scale bar: 100 μm. (D) Wright-Giemsa staining of the peripheral blood smear from moribund DKO mice and DWT control mice. Arrows indicate the blasts. Scale bar: 20 μm. (E) White blood cell, hemoglobin, and platelet count in the indicated mice. The mice in these groups were 12-14 months old. *p<0.05; **p<0.01; ***p<0.001.

FIGS. 2A-2F demonstrate that IL-6 signaling mediates MDS transformation to acute leukemia. (A) Complete blood cell counts of the indicated mice at the indicated time points. TWT, mDia1^{+/+}miR-146a^{+/+}IL-6^{+/+}, n=11; IL-6 KO, n=7; DKO, mDia1^{-/-}miR-146a^{-/-}IL-6^{+/+}, n=16; TKO, mDia1^{-/-}miR-146a^{-/-}IL-6^{-/-}, n=8. (B) Representative spleen images from the indicated mice (left) at 12-14 months of age. The spleen to body weight ratio was quantified (right). (C) Representative histology images of bone marrow and spleen from the indicated mice in B. The reticulin staining reveals increased fibrosis in DKO mice. The arrow and arrowhead indicate mitotic and apoptotic cells, respectively. Scale bars: 100 mm. (D) Kaplan-Meier survival analysis of the indicated mice. (E-F) In vitro colony-forming unit assay of nucleated cells from the bone marrow, spleen, and peripheral blood of the indicated mice at 12-14 months of age. Representative colonies are shown in E and quantified in F. Data are presented as Mean±SEM. *p<0.05; **p<0.01; ***p<0.001; **** p<0.0001.

FIGS. 3A-3G demonstrate that IL-6 deficiency ameliorates the defective hematopoiesis and leukemogenesis in DKO mice. (A) Percentages of the indicated cells in the peripheral blood mononuclear cells of the indicated mice at 12-14 months of age. TWT: n=12; IL-6 KO: n=8; DKO: n=10; TKO: n=10. Gran: granulocytes, Ly6G⁺CD11b⁺; MO: monocytes, Ly6C⁺CD11b⁺; B: B cells, B220⁺; T: T cells, CD3e⁺. (B) Flow cytometry plots illustrating the gating strategy for MDSCs in cells from A. gMDSC, granulocytic MDSC. (C) Quantification of MDSC cell populations in B. (D) Flow cytometric analyses of the expression levels of c-Kit among the indicated cell populations from C in the indicated mice. (E) Absolute number of cells in the indicated lineages were quantified in the bone marrow and spleens from indicated mice in A. (F) Cell size of indicated MDSCs in E were measured by flow cytometric forward scatter (FSC-A) and normalized to cells from TWT group. (G) Absolute number of erythroid cells in various developmental stages (I-VI) from the bone marrow and spleens of the indicated mice in C. The stages were determined by the cell surface expression levels of CD44. Stage I: proerythroblast; Stage II: basophilic erythroblast; Stage III: polychromatophilic erythroblast; Stage IV: orthochromatic erythroblast; Stage V: reticulocyte; Stage VI: mature erythrocyte. Data are presented as Mean±SEM. * p<0.05; ** p<0.01; *** p<0.001.

FIGS. 4A-4G show single-cell RNA-sequencing profiling that implicates IL-6 signaling in MDS transformation to AML with monocytic differentiation. (A) Merged Uniform Manifold Approximation and Projection (UMAP) plots from the bone marrow of 12-14 months old TWT, DKO, and TKO mice showing the distribution and overlap of annotated cell populations. (B) Same as A except the plots were shown separately for TWT, DKO, and TKO. (C) Merged UMAP plots from B highlighting the increased cell populations. (D) The percentages of the annotated cell types were compared among the indicated groups of mice. (E) KEGG pathway enrichment analysis of differentially expressed genes (DEGs) from A-D. The size of the circle represents the count of genes in each pathway. The color key from blue to red represents the low to high of adjusted p value based on −log 10. (F) Pairwise similarity analysis of selected cytokine levels across 4 groups of mice: TWT, IL-6 KO, DKO, and TKO. Darker red indicates co-expression patterns consistent within the 4 groups. (G) Hierarchically-clustering analyses of cytokine expression profiles from the serum of indicated mice determined by multiplex ELISA assay. Each column represents serum from one single mouse.

FIGS. 5A-51I show that IL-6 deficiency abolishes the transplantation abilities of leukemic initiating cells. (A) Schematic diagram of the transplantation strategies in B-D. (B) Kaplan-Meier survival analyses of CD45.1+ recipient mice transplanted with 2×10⁶ bone marrow mononuclear cells from the indicated mice. Both the recipient and donor mice were approximately 2 months old at transplantation. (C-D) Same as B except 2×10⁷ splenic mononuclear cells from moribund DKO mice or age-matched wild type counterparts were used as donor cells. The survival data before (C) and after (D) 21 days of transplantation are shown. (E) Complete blood counts of the recipient mice in D when the mice were 12 weeks post transplantation. (F) Wright-Giemsa staining of peripheral blood smear of mice in E. Scale bar: 20 μm. (G) Flow cytometric analysis evaluating the stem cell surface marker expression in peripheral blood from E. (H) Representative flow cytometric profiling of c-kit+ cells in the peripheral blood from mice in E. Data are presented as Mean±SEM. * p<0.05; ** p<0.01; *** p<0.001.

FIGS. 6A-6G demonstrates that IL-6 receptor and soluble IL-6 receptor are increased in the bone marrow of patients with high risk MDS. (A) IL-6 receptor mRNA levels were examined from a gene expression profiling dataset in CD34+ hematopoietic progenitor cells from patients with the indicated MDS subtypes. Control, n=17; MDS-EB1, MDS with excess blasts 1, n=37; MDS-EB2, MDS with excess blasts 2, n=43; MDS-RS, MDS with ring sideroblasts, n=48; MDS-SLD, MDS with single lineage dysplasia, n=55. (B) Kaplan-Meier analysis of overall survival in MDS patients with high or low expression levels of IL-6R from A. (C) Representative images of immunohistochemical staining of IL-6R in the bone marrow biopsies from patients with indicated MDS subtypes. Scale bar: 100 mm. (D) ELISA analyses of soluble IL-6R levels in the bone marrow (BM) aspirate or peripheral blood (PB) serum from control and MDS patients in a separate cohort from A. BM control, n=12; BM MDS, n=33; PB control, n=5; PB MDS, n=10. (E) ELISA analysis of soluble IL-6R of the bone marrow aspirate from different subtypes of MDS patients in D. Control, n=12; MDS-EB, n=15; MDS-MLD, MDS with multilineage dysplasia, n=6; MDS-RS, n=3; MDS-SLD, n=9. (F) ELISA analysis of soluble IL-6R in serum of the indicated mice at 12 months old. N=4 in each group. (G) Flow cytometric analyses of IL-6R expression on the cell surface of various cell lineages from indicated mice at 12 months old. Data are presented as Mean±SEM. * p<0.05; ** p<0.01; *** p<0.001.

FIGS. 7A-7D demonstrate that targeting IL-6 signaling ameliorates MDS to AML progression in the DKO model. (A) Schematic illustration of bone marrow transplantation. 5×10⁶ bone marrow cells from one-year-old DKO mice were transplanted into lethally irradiated one-year-old CD45.1+ recipient mice. (B) Kaplan-Meier survival analysis of the old CD45.1+ recipient mice transplanted with bone marrow cells from 1 year old DKO mice as illustrated in A. The mice were treated with the indicated reagents Once per week. n=7 in each group. (C) Complete blood cell counts of the mice from B administered with indicated reagents one-month post treatment. * p<0.05. (D) Representative H&E staining of the bone marrow, spleen, and liver from the mice in B. Scale bars: 100 mm.

FIGS. 8A-8F demonstrate that tocilizumab reduces cell proliferation and colony formation in MDS patient cells. (A) Cultured MDS-derived cell line (MDSL) cells were treated with tocilizumab or IgG for 1 hour at the indicated concentration. Cells were then challenged with human recombinant IL-6 (10 ng/mL) for 15 min followed by a western blot assay of p-STAT3. Actin was used as a loading control. (B) 1×10⁴/well MDSL cells were seeded in a 96-well plate on day 0 in MDSL culture medium with 50 μg/mL tocilizumab or 50 μg/mL human IgG control. Relative cell number was assessed with CCK-8 reagent at the indicated time points. (C) 1×10⁶ MDSL cells were transplanted into sub-lethally irradiated NSG recipient mice. 10 days after transplantation, mice were subjected to weekly treatment with tocilizumab (TCZ) or human IgG (8 mg/kg) by intraperitoneal administration. Engraftment was evaluated 60 days post transplantation via flow cytometry assays of hCD45+ mononuclear cells in the peripheral blood. N=5 in each group, data are presented as Mean±SD. (D) Quantification of the percentage of hCD45+ cells in C. (E-F) Colony-forming unit (CFU) assays in normal (E) and high risk MDS patient (F) bone marrow derived CD34+ cells. 1×10³ normal (E) or 2×10³ patient CD34+ cells (F) were seeded in MethoCult medium supplemented with human IgG or tocilizumab (50 μg/mL) on day 0. The number of colonies was accessed on day 14. Triplicate assay colonies were independently identified by two individuals. Data are presented as Mean±SD. E, G, M, GM, and GEMM represent BFU/CFU-E, CFU-G, CFU-M, CFU-GM, and CFU-GEMM, respectively, in both E and F.

FIGS. 9A-9D demonstrate that loss of IL-6 rescues multi-organ leukemia infiltrations in DKO mice. (A) Microarray gene expression data of bone marrow CD34+ cells from 47 MDS patients. Housekeeping genes GAPDH and ACTB are included to demonstrate the modest raw intensity without normalization. TNNT2 and CD34 were plotted as a negative and positive control, respectively. Data is from GSE160727. (B) Representative gross images of the lungs from the indicated mice at the age of 12-14 months. Arrows indicate tumors. (C) Representative images of H&E staining of the lung tissues from A. Arrows indicate the blasts present in DKO mice. Scale bars: 100 μm. (D) Representative images of H&E staining and reticulin staining (bottom row) of the liver tissues from the indicated mice at the age of 12-14 months. Arrows in the DKO panel indicate blast infiltration. The dotted lines in the DKO panels separate normal and neoplastic tissues. Scale bar: 100 μm.

FIG. 10 shows the gating strategy used for the identification of different erythroblast populations.

FIG. 11 is a heatmap showing the expression levels of marker genes for each cell population identified in the TWT mice studied in FIGS. 4A-4D.

FIG. 12 is a heatmap showing the expression levels of marker genes for each cell population identified in the DKO mice studied in FIGS. 4A-4D.

FIG. 13 is a heatmap showing the expression levels of marker genes for each cell population identified in the TKO mice studied in FIGS. 4A-4D.

FIG. 14 shows the loss of IL-6 reverted genes that are upregulated in the macrophage group in DKO bone marrow. Differentially expressed genes (DEG) identified in the macrophages of TWT and DKO mice are illustrated in a Venn diagram on the left. The results of a KEGG pathway analysis of common DEG is shown on the right.

FIGS. 15A-15B demonstrate that the transplantable leukemia from DKO mice is derived from the c-Kit positive cells. (A) Representative bone marrow sections of recipient mice at 5 months after transplantation of spleen c-Kit+ cells from 12-month-old DKO mice. Mice in the wild-type control group were transplanted with bone marrow c-Kit+ cells from 12-month-old TWT mice. Scale bar: 20 μm. (B) Same as A except spleen sections were analyzed in the indicated mice. H&E staining, scale bars: 100 μm.

FIGS. 16A-16C demonstrate that inhibition of IL-6 signaling does not affect early MDS phenotypes in DKO mice. (A) Total bone marrow cells from wild-type C57/BL6 mice were collected in serum-free RPMI1640 medium with anti-mouse IL-6 antibody (left) or mouse gp130-Fc chimera (right). After 1 hour incubation, cells were challenged with mouse recombinant IL-6 for 15 minutes before RIPA lysis for western blot analyses of the indicated proteins. (B-C) 5-month-old recipient mice were transplanted with bone marrow cells from 5-month-old DKO mice. The recipient mice were treated with Anti-HRP IgG control, gp130-Fc chimera, or anti-IL-6 antibody once every week. Complete blood counts were analyzed at 1 month (B) and 4 month (C) post-treatment. Data presented in Mean±SD. P values are listed above the plots.

FIGS. 17A-17B demonstrate that tocilizumab partially induces cell death in MDSL cells (A) and reduces spleen weight in a MDSL xenograft model (B).

### DETAILED DESCRIPTION

The present disclosure provides methods of using an inhibitor of the IL-6 signaling pathway to inhibit disease progression in a subject with high-risk myelodysplastic syndromes (MDS), reduce the growth of blasts, or treat low blast count acute myeloid leukemia (AML). Also provided is use of an animal model of MDS to AML progression for identifying an inhibitor of MDS to AML progression.

Myelodysplastic syndromes (MDS) are age-related myeloid neoplasms that can progress into acute myeloid leukemia (AML). However, the mechanisms of MDS to AML progression are poorly understood, especially in relation to the aging microenvironment. To study this process, the inventors studied a mDia1/miR-146a double knockout (DKO) mouse model that phenocopies MDS. These mice develop age-related pancytopenia with over-secretion of pro-inflammatory cytokines. As described in the Examples, the inventors found that most of the DKO mice underwent leukemic transformation at 12-14 months of age. These mice showed myeloblast replacement of fibrotic bone marrow and widespread leukemic infiltration. Strikingly, depletion of IL-6 in these mice largely rescued the leukemic transformation and markedly extended survival. Single-cell RNA sequencing analyses revealed that DKO leukemic mice had an increased number of monocytic blasts that were reduced with IL-6 knockout. The studies further revealed that the levels of IL-6 receptor (IL-6R) in the bone marrow were significantly increased in high risk MDS patients and in older DKO mice. Blocking of IL-6 signaling significantly ameliorated AML progression in the DKO model and clonogenicity of CD34 positive cells from MDS patients. Thus, this work establishes a mouse model of age-related MDS to AML progression and suggests that treatments that target IL-6 signaling may be effective against high risk MDS.

The methods of the present disclosure, in which an inhibitor of the IL-6 signaling pathway is administered to treat MDS or AML, offer several advantages over the current standard of care, i.e., chemotherapy and/or bone marrow transplantation. First, the side-effects of IL-6 signaling pathway inhibitors are far less severe than those of chemotherapy, making these methods more tolerable for patients who are elderly or respond poorly to chemotherapy. Second, IL-6 signaling pathway inhibitors have undetectable inhibitory effects on normal hematopoietic progenitor stem cells (HPSCs) and are specific to malignant HPSCs.

Methods of Inhibiting Progression of Myelodysplastic Syndromes

In a first aspect, the present disclosure provides a method of inhibiting disease progression in a subject with a high-risk myelodysplastic syndrome (MDS). The method comprises administering an inhibitor of the IL-6 signaling pathway in an amount effective to inhibit disease progression.

“Myelodysplastic syndromes (MDS)” are a group of cancers in which immature blood cells in the bone marrow (i.e., blasts) fail to mature into healthy blood cells. As a result, the bone marrow produces too few mature, functional red blood cells, white blood cells, or platelets. Symptoms of MDS include fatigue, shortness of breath, bleeding disorders, anemia, and frequent infections. Risk factors for MDS include previous chemotherapy or radiation therapy, exposure to certain chemicals (e.g., tobacco smoke, pesticides, benzene), and exposure to heavy metals (e.g., mercury, lead).

There are several different types of MDS. For example, the World Health Organization (WHO) recognizes 6 main types of MDS: MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassifiable (MDS-U). Some types of MDS, referred to as “low-risk MDS,” progress slowly and may cause mild to moderate anemia (i.e., a low number of red cells) or decrements to other types of cells. Other types of MDS, referred to as “high-risk MDS” (e.g., MDS-EB), can cause severe problems. In patients with high-risk MDS, immature blood cells referred to as “blasts” make up more than 5 percent of the cells in the bone marrow. The excess blasts do not develop into normal blood cells, which causes severe deficits. Specifically, low blood cell counts can lead to anemia (i.e., low red cell count), neutropenia (i.e., low neutrophil count) or thrombocytopenia (i.e., low platelet count).

MDS and acute myeloid leukemia exist along a continuous disease spectrum. When MDS patients develop more than 20% blast cells, they are reclassified as having acute myeloid leukemia. “Acute myeloid leukemia (AML)” is a cancer of the myeloid line of blood cells. Patients with AML developed from MDS generally have a poor prognosis, as this cancer often progresses rapidly and can be fatal within weeks or months if left untreated.

In addition to progressing into AML, MDS may also progress into life-threatening bone marrow failure. “Bone marrow failure” occurs when the bone marrow fails to produce enough healthy blood cells to keep up with the body's needs.

Thus, as used herein, the phrase “inhibiting disease progression” includes reducing, repressing, delaying, or preventing progression of MDS into AML, development of AML, and/or bone marrow failure compared to an untreated control. This phrase also includes reducing, repressing, delaying, inhibiting, or preventing cancer growth, and reducing the number of cancer cells within a subject compared to an untreated control. Accordingly, the phrase “amount effective to inhibit disease progression” refers to an amount that is sufficient to achieve one or more of these outcomes. For any active agent, an effective amount can be estimated initially in cell culture assays or in animal models.

As used herein, an “untreated control” is a subject that is comparable to the subject being treated (e.g., of the same species, breed, and sex, and of a similar age) that was raised under the same or comparable conditions (e.g., diet, environment) but was not administered an inhibitor of the IL-6 signaling pathway.

In some embodiments, these outcomes result in extended survival of the subject compared to an untreated control. In some embodiments, the survival of the subject is extended by at least about 5%, 10%, 12%, 15%, 20%, or 25% compared to the untreated control.

The “subject” to which the methods of the present invention are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In a preferred embodiment, the subject is a human.

The method may reduce the growth of blasts. Myeloid cells are differentiated descendants from common myeloid progenitors derived from hematopoietic stem cells in the bone marrow. Myeloid cells broadly include erythroid cells, granulocytes, monocytes, macrophages, dendritic cells, and megakaryocytes. In MDS and AML, the bone marrow produces “blasts”, which are abnormal immature myeloid progenitor cells that are ineffective in producing mature blood cells. In comparison to a mature blood cell, a blast cell has one or more of the following morphological characteristics: a large nucleus, immature chromatin, a prominent nucleolus, scant cytoplasm, and few or no cytoplasmic granules.

The phrase “reducing the growth of blasts” includes reducing the number of blasts. In some embodiments, the number of blasts is reduced by at least about 5%, 10%, 12%, 15%, 20%, or 25% compared to an untreated control.

The blasts may be CD34+ cells. “CD34” (i.e., cluster of differentiation 34) is a transmembrane phosphoglycoprotein that is a marker of hematopoietic stem cells and progenitor cells. All colony-forming cells in human bone marrow comprise this surface marker. CD34+ cells from MDS patients harbor genetic abnormalities that are not found in CD34+ cells from healthy individuals.

The blasts may be CD34+ negative cells that are morphologically defined as blasts.

The blasts found in MDS patients are more sensitive to the inhibition of the IL-6 pathway compared to their normal counterparts. In the Examples, the inventors demonstrate that treating MDS-derived CD34+ cells with an inhibitor of the IL-6 signaling pathway (i.e., tocilizumab) reduces the colony numbers derived from the CD34+ cells found in MDS patients. However, tocilizumab has no effects on the colony numbers derived from normal CD34+ cells. Thus, in some embodiments, the methods result in selective killing of blasts in MDS patients.

Methods of Treating Low Blast Count Acute Myeloid Leukemia

In a second aspect, the present disclosure provides a method of treating low-blast count AML in a subject. The AML may have developed from MDS. The method comprises administering an inhibitor of the IL-6 signaling pathway in an amount effective to treat the AML.

“Low blast count acute myeloid leukemia (AML)” is a subtype of AML in which blasts make up 20-30% of the bone marrow. This subtype is characterized by peculiar features, such as increased frequency in elderly individuals and after cytotoxic therapies, poor-risk cytogenetics, and lower white blood cell counts. The clinical course of this subtype is often similar to that of MDS with 10-19% blasts.

As used herein, “treating” describes the management and care of a subject for the purpose of combating low blast count AML. Treating includes the administration of an inhibitor of the IL-6 signaling pathway to prevent the onset of symptoms or complications, to alleviate the symptoms or complications, or to eliminate the disease, condition, or disorder. Accordingly, the phrase “amount effective to treat the AML” refers to an amount sufficient to achieve one or more of these outcomes. This phrase also includes reducing, repressing, delaying, inhibiting, or preventing symptoms of AML, and reducing the number of blasts within a subject compared to an untreated control.

All the methods disclosed herein involve administering an inhibitor of the IL-6 signaling pathway. “Interleukin-6 (IL-6)” is a multifunctional proinflammatory cytokine. MDS blasts depend on IL-6 for survival, proliferation, and progression. In the Examples, the inventors demonstrate that treatment with inhibitors of the IL-6 signaling pathway reduces MDS to AML progression and reduces the growth of blasts. Thus, inhibitors of the IL-6 signaling pathway may be used to specifically target malignant cells that depend on IL-6 signaling to treat high risk MDS.

Initiation of the IL-6 signaling pathway occurs when IL-6 binds to its receptor, which is referred to as interleukin 6 receptor (IL-6R; also known as CD126). IL-6 binding allows IL-6R to associate with glycoprotein 130 (gp130; also known as CD130). Gp130 then undergoes homo-dimerization and signal transduction. Thus, inhibitors of the IL-6 signaling pathway include antagonists for IL-6, IL-6R, gp130, and downstream signaling molecules. Four inhibitors of IL-6 signaling are commercially available for clinical use: tocilizumab, sarilumab, and satralizumab, which are all monoclonal antibodies directed against IL-6R, and siltuximab, a monoclonal antibody specific for IL-6. Thus, the inhibitor is an anti-IL-6 or anti-IL-6R antibody. In exemplary embodiments, the inhibitor is tocilizumab. In other exemplary embodiments, the inhibitor is a recombinant gp130 Fc chimera protein that can neutralize IL-6 to prevent IL-6-mediated signaling.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, intraperitoneal administration, and subcutaneous administration. Administration can be continuous or intermittent. In some embodiments, the inhibitor is administered once a week.

Use of Animal Models of Myelodysplastic Syndromes Progression

In a third aspect, the present invention provides a method for identifying a therapeutic for preventing MDS to AML progression. The method comprises transplanting bone marrow cells from a mDia1/miR-146a double knockout (DKO) animals having MDS into a test animal, administering a candidate therapeutic to the test animal, and measuring the progression of MDS to AML in the test animal, wherein a decrease in the progression of MDS to AML in the test animal compared to an untreated control animal transplanted with bone marrow cells from the DKO animal identifies the therapeutic. As used herein, an “animal model” is an animal that has been modified by the hand of man such that it mimics aspects of a disease found in humans. Any non-human animal may be used as an animal model. Suitable animal models include, for example, mice, rats, rabbits, guinea pigs, hamsters, gerbils, zebrafish, dogs, pigs, and non-human primates. In preferred embodiments, the animal model is a mouse model. As used herein, “therapeutic” refers to an agent or compound that relieves to some extent one or more signs, symptoms, or causes of a disease or condition.

The DKO animal models of the present invention have been modified such that they have reduced expression of mDia1 and miR-146a compared to a wild-type animal. mDia1 is a member of the formin protein family and is a Rho effector. miR-146a is a miRNA expressed in hematopoietic stem cells that is known for its ability to dampen immune responses. Expression of these gene products can be reduced in several ways, including by “knocking them out” by replacing them or disrupting them with artificial DNA (e.g., using a CRISPR-based technology or traditional embryonic stem cell-based technology) or by “knocking them down” by expressing an RNA molecule (e.g., small-interfering RNA (siRNA), short hairpin RNA (shRNA), anti-sense RNA, and microRNA (miRNA)) that interferes with their expression or function. In the Examples, the inventors demonstrated that a mDia1/miR-146a double knockout mouse can be used as an animal model of MDS to AML progression. As used herein, a “wild-type animal” is an animal that has expressed mDia1 and miR-146a at “normal” levels.

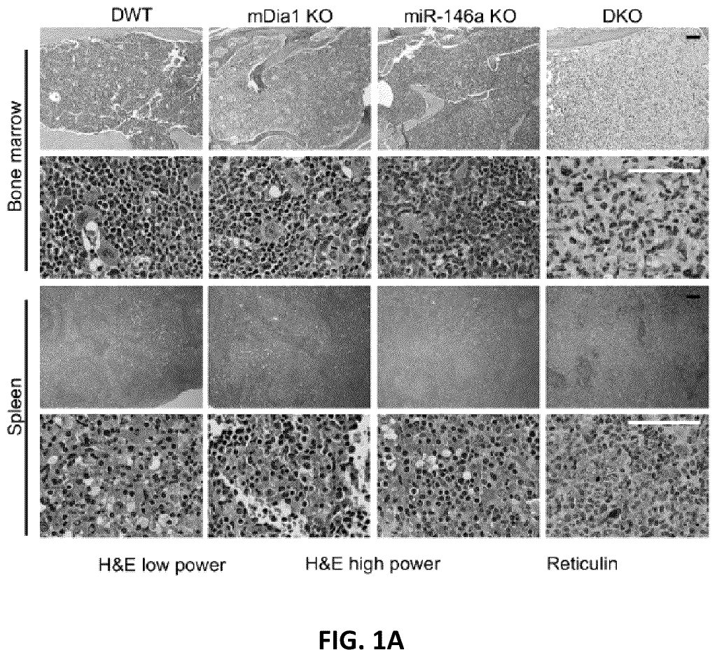

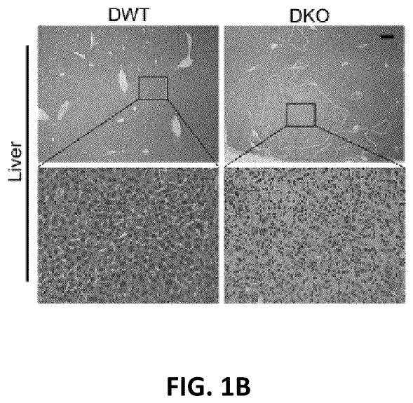

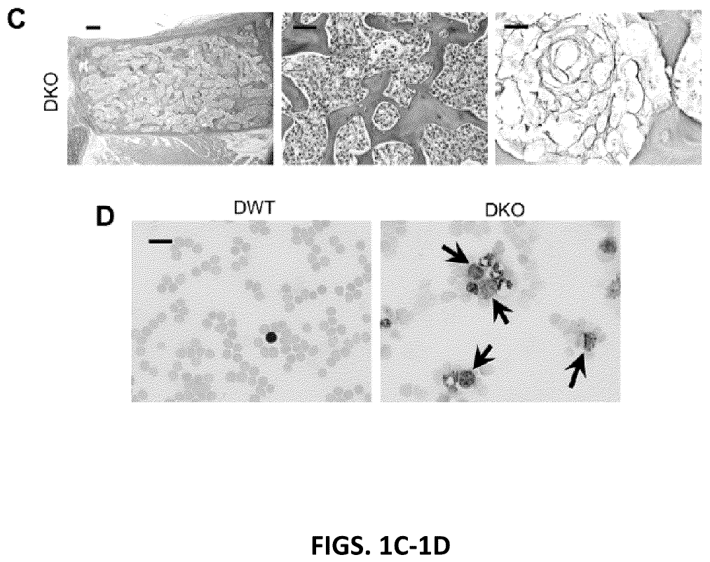

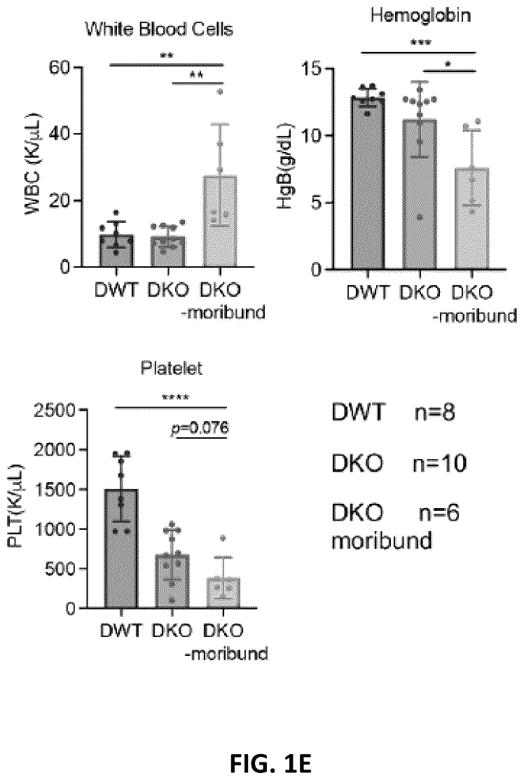

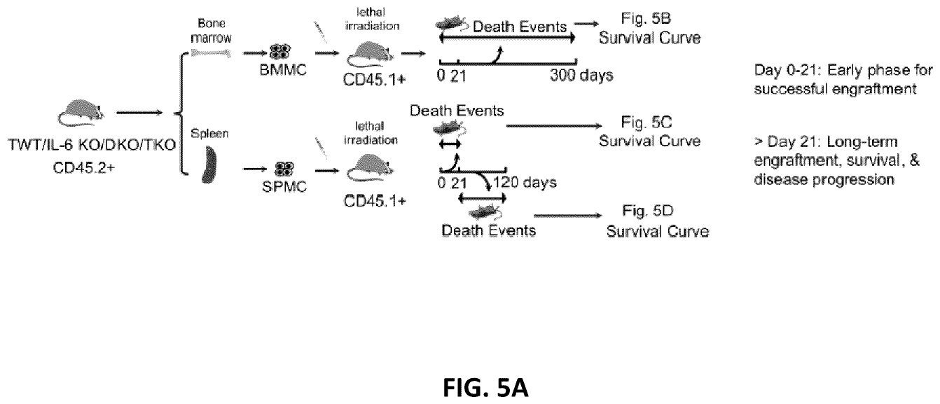

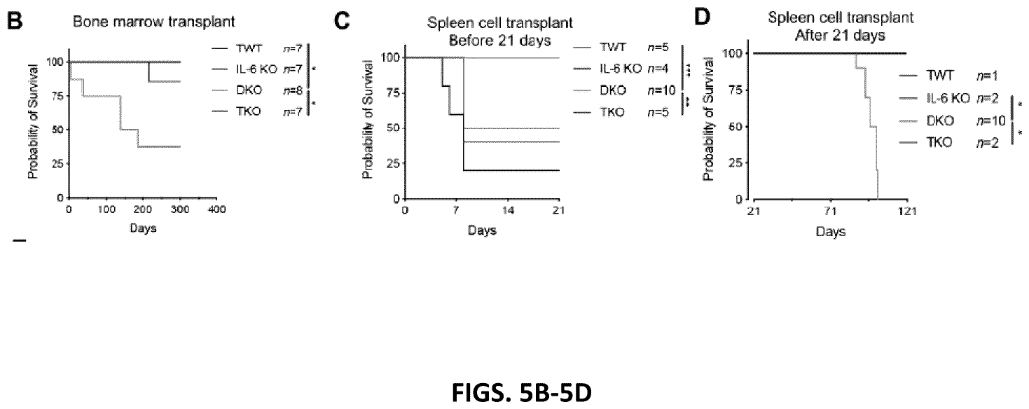

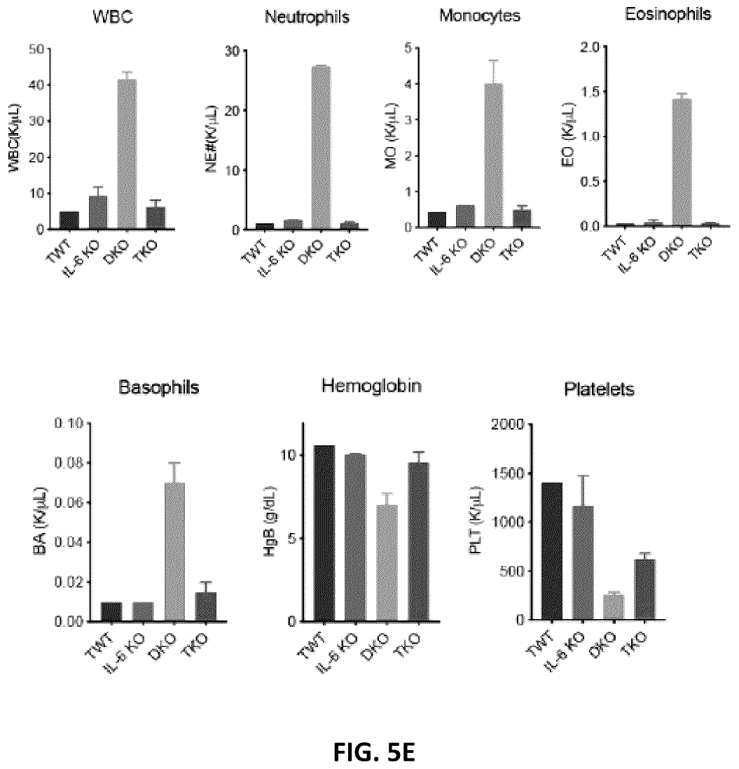

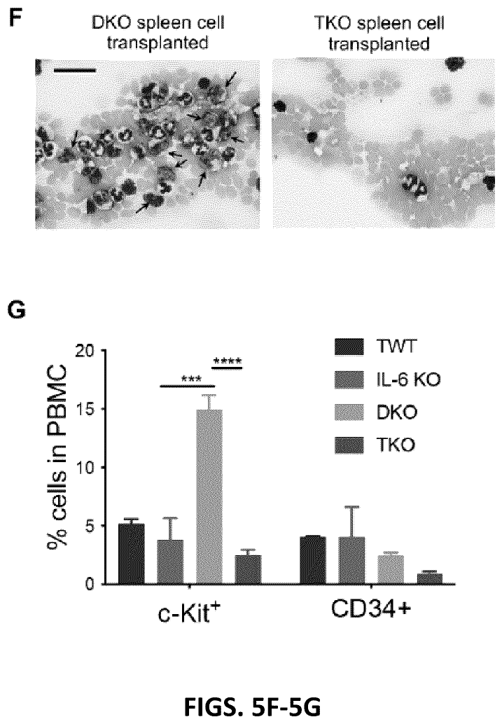

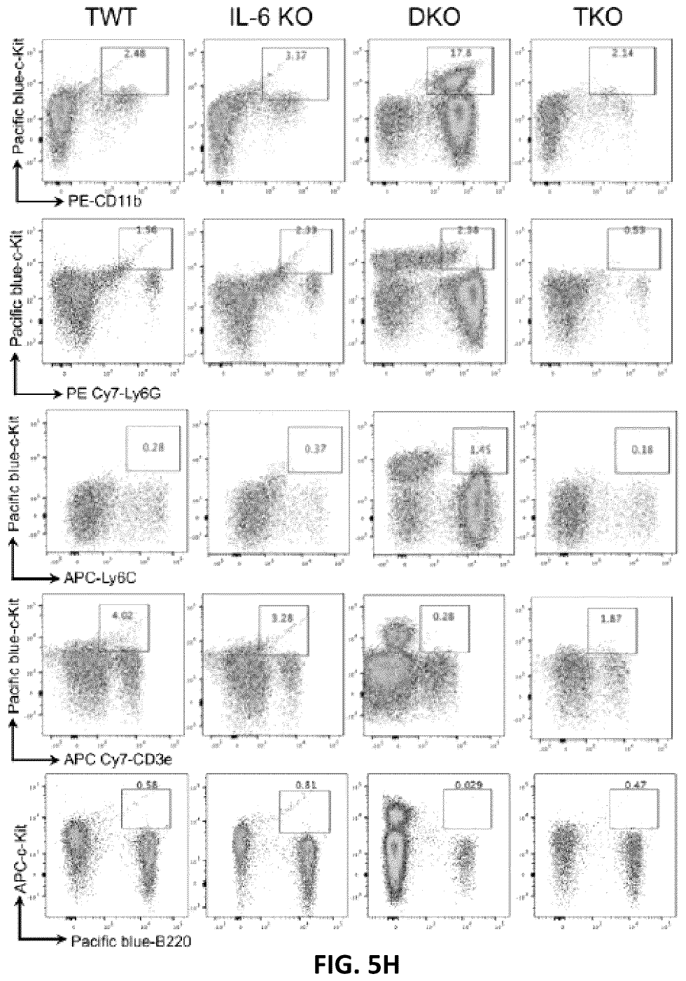

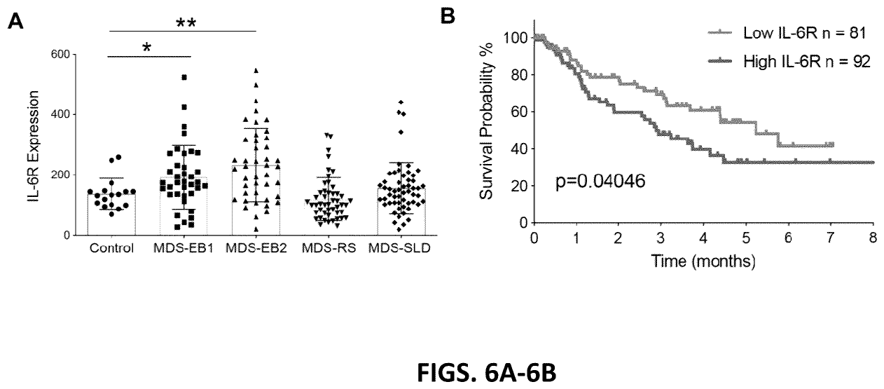

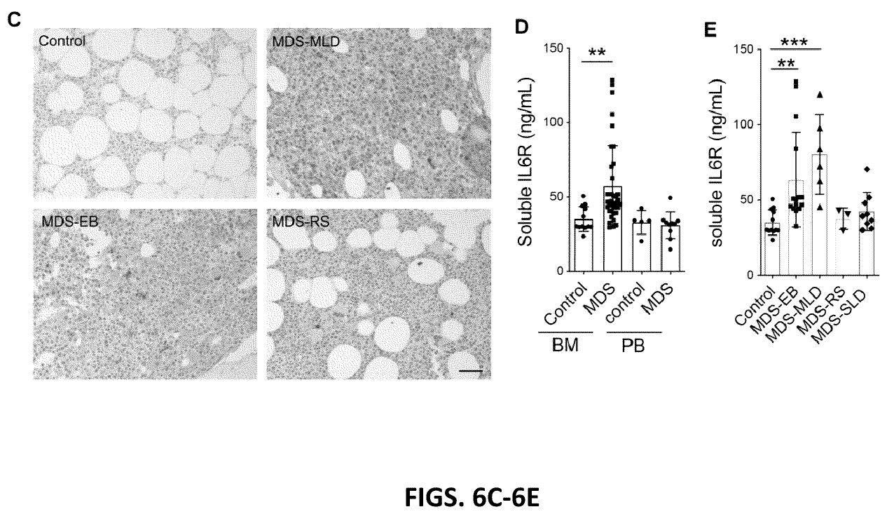

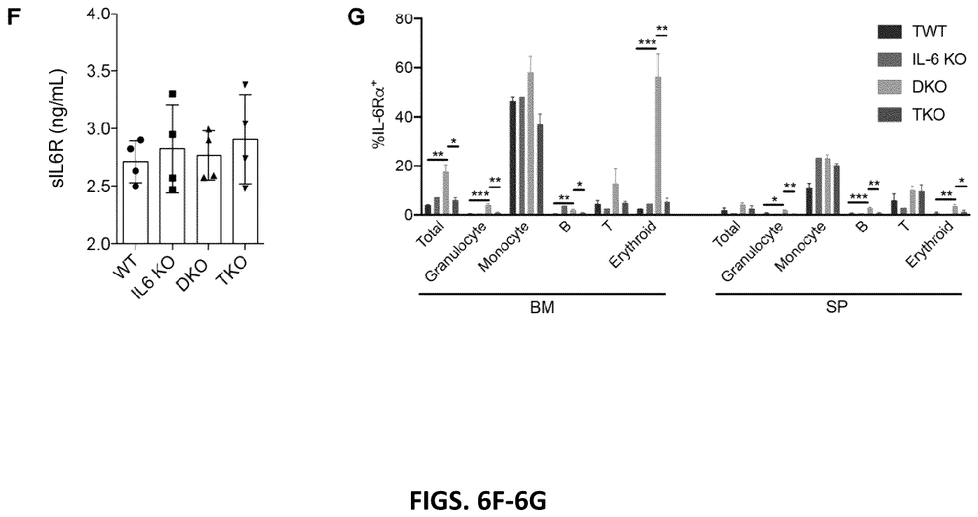

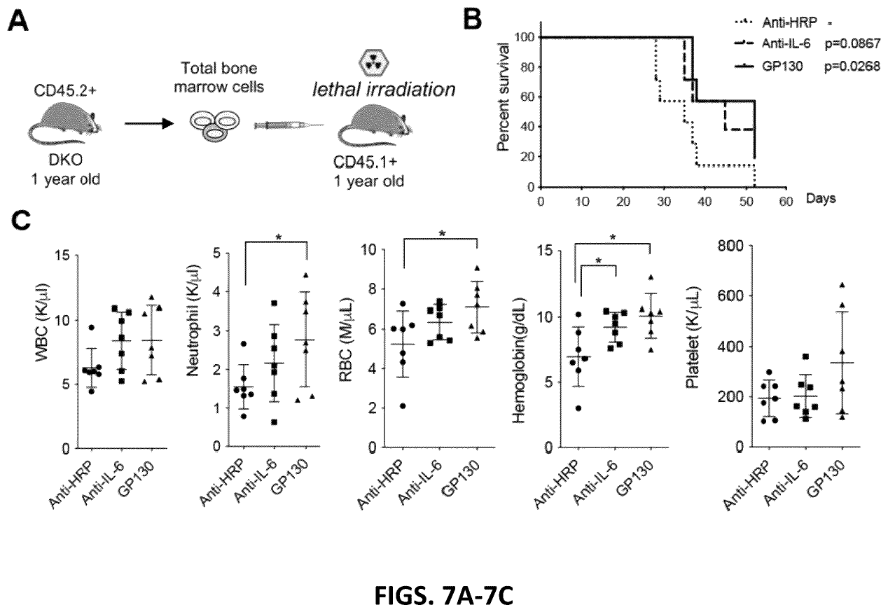

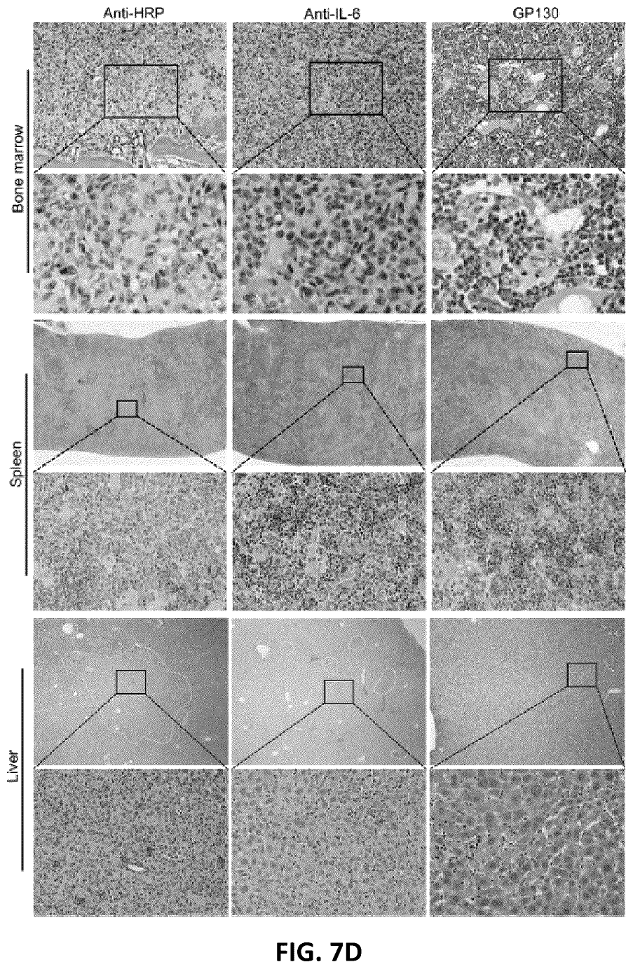

In the Examples, the inventors show that their mDia1/miR-146a double knockout (DKO) mice express increased levels of cell surface and soluble IL-6R in the bone marrow compared to wild-type mice (see FIG. 6). Thus, in some embodiments, the animal model expresses increased levels of IL-6R in the bone marrow compared to a wild-type animal. The inventors show that transplanting bone marrow cells from their mDia1/miR-146a double knockout (DKO) mouse model into recipient mice phenocopies MDS to AML progression. Specifically, they demonstrate that transplantation of bone marrow or leukemic splenic cells from the DKO mice into a lethally irradiated recipient animal causes the recipient mice to develop MDS (see FIG. 5). Thus, the test/recipient animal may be irradiated before transplantation. The inventors show that the recipient mice exhibit pancytopenia, replacement of normal hematopoiesis by blasts in the bone marrow, infiltration of the spleen by blasts, and/or infiltration of the liver by blasts (see FIGS. 1 and 7).

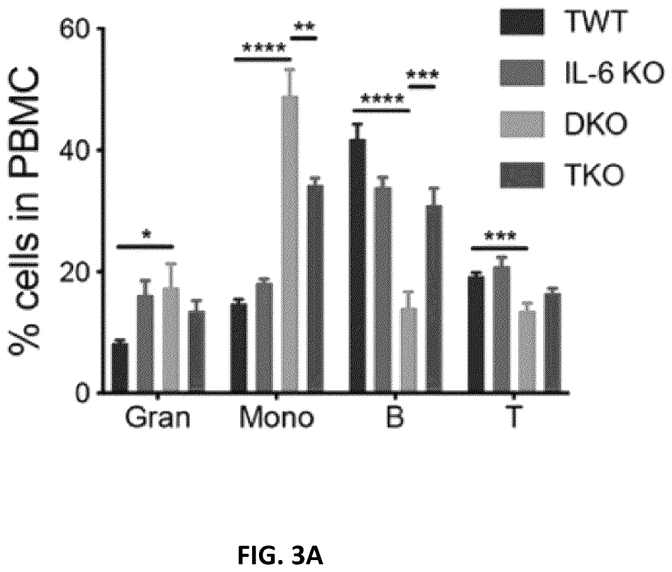

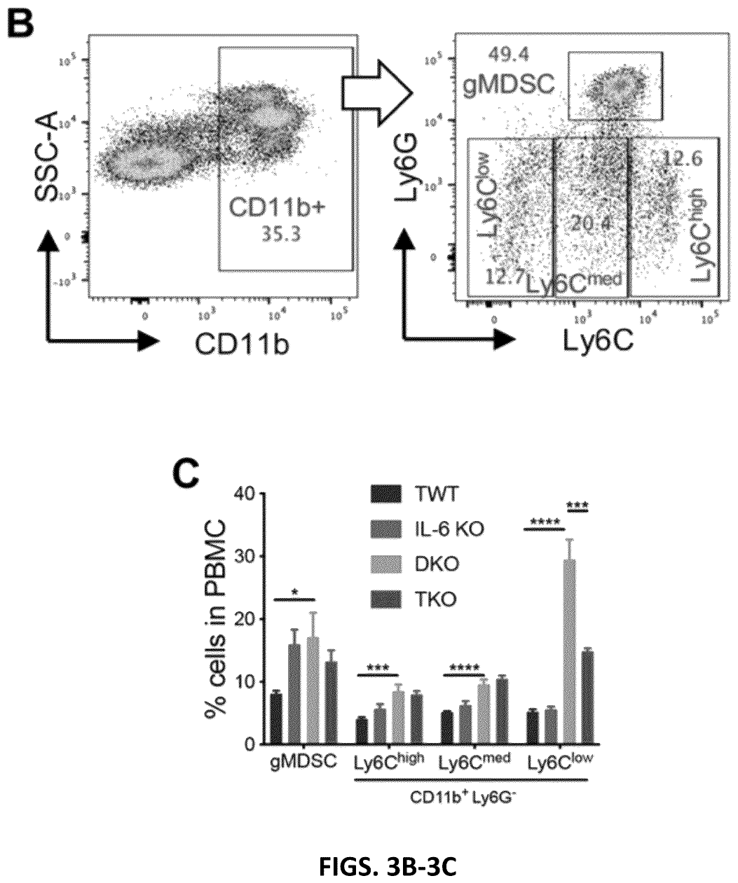

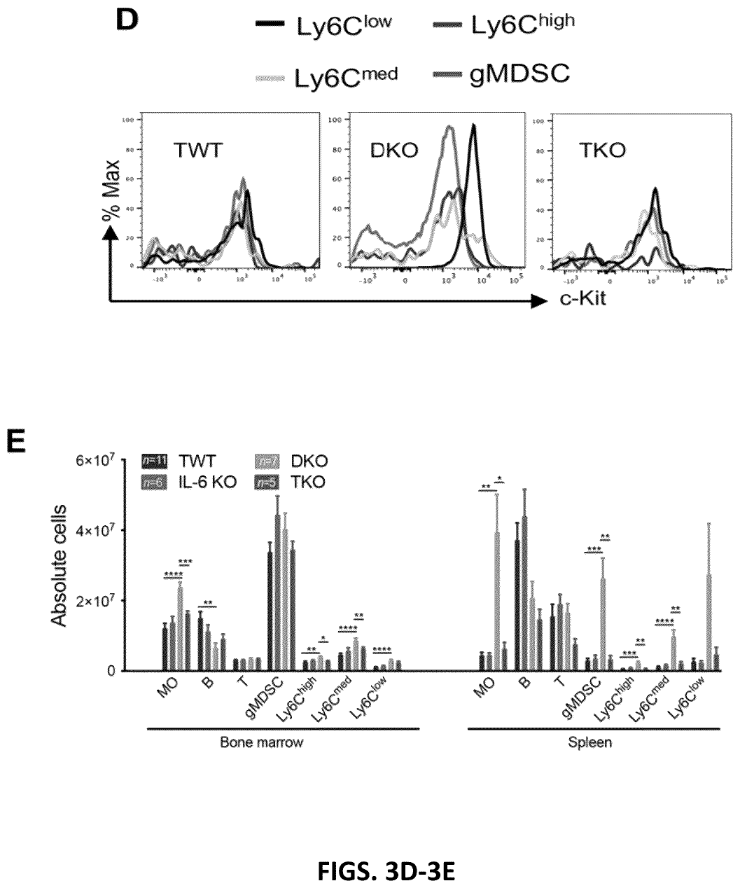

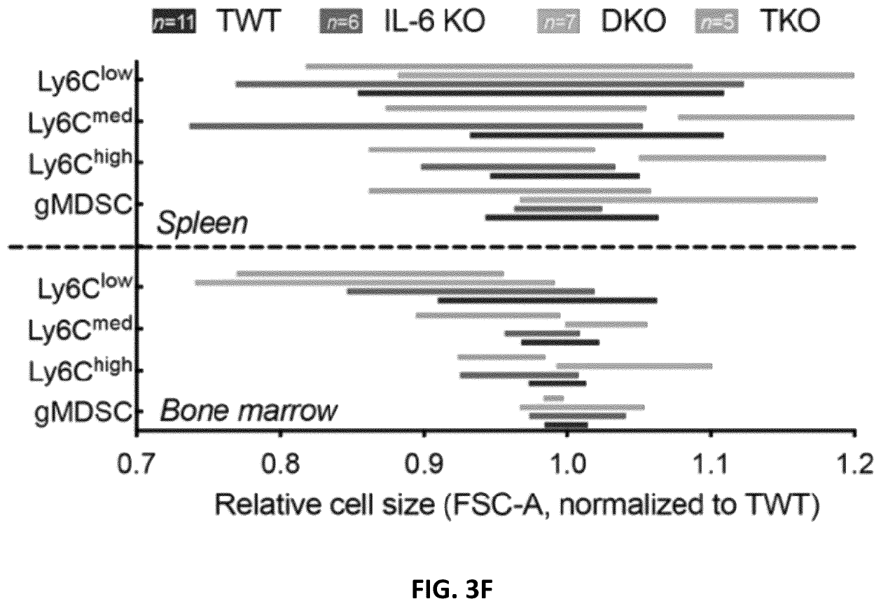

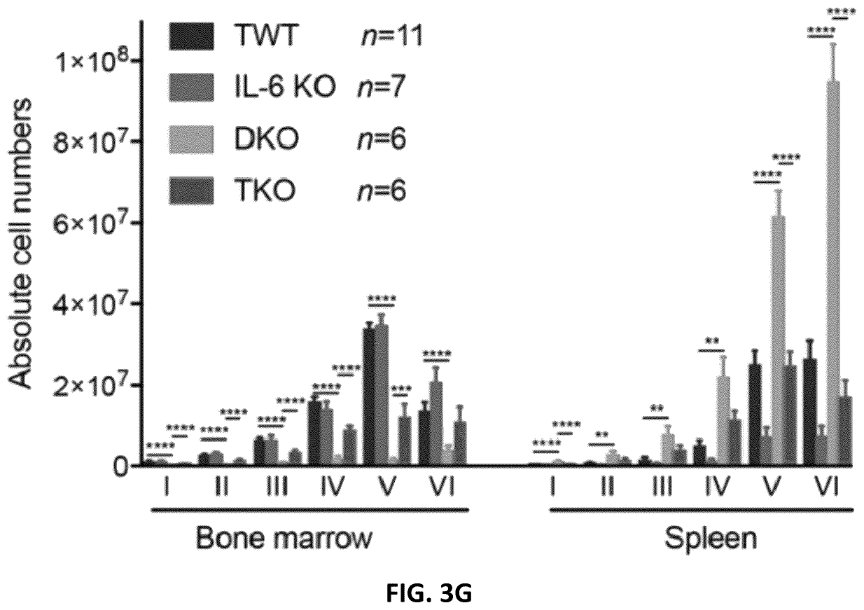

Notably, the inventors chose these tissues for use in their transplant experiments because MDS blasts accumulate in the bone marrow and the spleen. In the Examples, the inventors demonstrated that the transplantable leukemia from their mDia1/miR-146a double knockout mice is derived from the c-Kit positive cells (see FIG. 3). c-Kit is a receptor tyrosine kinase that is involved in intracellular signaling and a commonly used marker of blasts. Thus, in some embodiments, the transplanted bone marrow or splenic cells are c-Kit positive.

A decrease in progression of MDS to AML may be determined by increased survival of the test animal compared to the untreated animal. A decrease in progression of MDS to AML may also be determined by reduced pancytopenia in the test animal compared to the untreated animal. Pancytopenia is a condition in which there is a lower than normal number of red and white blood cells and platelets in the blood. Thus, pancytopenia may be measured by reduced white blood cells, neutrophils, red blood cells, platelets, etc.

A decrease in progression of MDS to AML may also be determined by reduced growth of blasts in the test animal compared to the untreated animal. The identification and/or measurement of blasts includes observing the number of blasts in a tissue or in circulation. This may be performed by hematoxylin and eosin (H&E) staining of fixed tissue samples and/or May-Grunwald-Giemsa staining of peripheral blood or bone marrow smears, as shown in the Examples, herein. However, any methods of measuring blasts known in the art may be used.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Example

Introduction:

Myelodysplastic syndromes (MDS) are age-related clonal myeloid neoplasms characterized by ineffective hematopoiesis. Patients with high risk MDS have a significantly increased risk of progression into acute myeloid leukemia (AML). The prognosis of AML developed from MDS is poor with limited treatment options. Genetically, recurrent chromosome abnormalities, including del(5q), loss of chromosome 7, and del(7q), are frequently detected in MDS patients (1, 2). MDS has a complex molecular pathophysiology, as evidenced by the fact that somatic mutations are found in over 40 genes in this disease, including SF3B1, TET2, ASXL1, DNMT3A, and TP53 (3). These mutations were also found in low allele frequency in apparently healthy old individuals with clonal hematopoiesis of indeterminate potential (CHIP), who have an increased risk of developing MDS (4, 5). An age-related inflammatory bone marrow microenvironment is also involved in the development of the disease (6-13).

Many animal models have been developed to phenocopy MDS, including models comprising xenotransplants of hematopoietic cells from patients with MDS and genetically modified mice harboring mutations found in MDS (14). However, each of these types of models has its drawbacks: the xenotransplantation models suffer from poor engraftment efficiency, and genetically modified mice fail to reflect the complex genetic abnormalities of MDS. Nevertheless, AML development has been seen in NUP98-HOXD13 (NHD13) hematopoietic specific transgenic mice (15), NPM1 haploinsufficiency mice (16), BCL-2 and mutant NRAS co-expression mice (17), RUNX1 mutant mice (18), and Arid4a deficient mice (19). These models are valuable tools for the investigation of potential therapeutic agents for treating MDS and for preventing MDS to AML progression. However, the contributions of the bone marrow microenvironment to AML progression cannot be elucidated from these models.

We have previously shown that loss of DIAPH1, a gene located on 5q31 in humans and involved in the regulation of actin polymerization (20-22), led to an aberrant overexpression of CD14 on Gr1/Mac1 double positive granulocytes and activation of Toll-like receptor 4 (TLR4) through pathogen-associated molecular patterns (PAMPs) and damage-associated molecular patterns (DAMPs) in mice (6). More recently, we explored the innate immune pathway and inflammation in MDS using a mouse model with concurrent deletion of mDia1 (encoded by Diap1 in mice) and miR-146a (23). MIR146A is also located on chromosome 5q in humans and is involved in repression of the TLR-TRAF6 pathway (24-26). Studies using miR-146a knockout mice show that miR-146a serves as a brake on inflammation and regulates myeloproliferation and oncogenic transformation (25). Therefore, the mDia1/miR-146a double knockout (DKO) mice more closely mimic MDS patients in that they have an inflammatory bone marrow microenvironment. We demonstrated that mice with constitutive knockout of both mDia1 and miR-146a show age-related pancytopenia and are hypersensitive to aging-associated accumulation of DAMPs and PAMPs. This leads to an increased production of proinflammatory cytokines by myeloid-derived suppressive cells (MDSCs). Pathologic levels of these cytokines are detrimental to terminal erythropoiesis leading to increased cell death, which provides more DAMPs and forms a positive feedback loop to further worsen the inflammatory environment in these mice. These data together reveal the critical role of the inflammatory bone marrow environment in MDS pathogenesis.

In the following study, we found that the DKO mice develop AML when moribund at the age of 12-14 months. We further reveal that bone marrow-confined IL-6 signaling plays a pivotal role in MDS to AML progression in the DKO mouse model, which is consistent with MDS patient data. Our study suggests that targeting the IL-6 signaling pathway will benefit high risk MDS patients by preventing AML progression.

Materials and Methods:

Animals

The mDia1/miR-146a double knock out mice have been described previously (23). In brief, miR-146a^{-/-} mice in C57/BL6 background purchased from the Jackson Laboratory (stock No. 016239) were crossed with mDia1 deficient mice to generate mDia1^{-/-}miR146a^{-/-}mice (DKO mice) (6). To generate mDia1^{-/-}miR146a^{-/-}IL-6^{-/-} triple knockout mice (TKO), IL-6 knockout mice purchased from the Jackson Laboratory (stock No. 002650) were crossed with DKO mice. The CD45.1 congenic mice were purchased from Charles River (B6-LY-5.12/Cr, strain code: 564). All the experiments were conducted in accordance with the Guide for the Care and Use of Laboratory Animals and approved by the Institutional Animal Care and Use Committees at Northwestern University.

Reagents

The detailed reagent information is listed in Table 1, below.

TABLE 1
Antibodies used for flow cytometric assays and immunohistochemistry staining.

| | | |
|---|---|---|
| | | Supplier or |
| Antibody | Catalog number | comments |

Antibodies for flow cytometric assay

| | | |
|---|---|---|
| PE-CD11b | M1/70, Cat# 553311 | BD Pharmingen |
| PE-Cy7-Gr1 | RB6-8C5, Cat# 108416 | BioLegend |
| Pacific Blue-B220 | RA3-6B2, Cat# 103227 | BioLegend |
| APC-eFluor780-CD3e | 17A2, Cat# 47-0032-82 | eBioscience |
| APC/Fire 750-CD3e | 17A2, Cat# 100248 | BioLegend |
| PE-Cy7-Ly6G | 1A8, Cat# 560601 | BD Pharmingen |
| APC-Ly6C | HK1.4, Cat# 128016 | Biolegend |
| APC-TER-119 | TER-119, Cat# 17-5921-82 | eBioscience |
| PE-CD44 | IM7, Cat# 12-0441-82 | eBioscience |
| BV421-CD34 | RAM34, Cat# 562608 | BD Horizon |
| PE-Sca1(Ly-6A/E) | D7, Cat# 108108 | BioLegend |
| PE-Cy7-CD117(c-Kit) | 2B8, Cat# 105814 | BioLegend |
| Pacific Blue-CD117(c-Kit) | 2B8, Cat# 105820 | BioLegend |
| APC-CD117(c-Kit) | 2B8, Cat# 17-1171-82 | eBioscience |
| APC anti-mouse CD126 (IL- | D7715A7, Cat# 115812 | Biolegend |
| 6Rα chain) | | |
| BV421 anti-human CD45 | 2D1, Cat# 368521 | Biolegend |

Antibodies for immunohistochemistry staining

| | | |
|---|---|---|
| Anti-Human IL-6R | Cat # PA5-102425 | Invitrogen |

Antibodies for western blotting

| | | |
|---|---|---|
| Anti-Stat3 rabbit mAb | Cat# 12640 | Cell Signaling |
| | | Technology |
| Anti-pStat3 (Tyr705) rabbit | Cat# 9145 | Cell Signaling |
| mAb | | Technology |
| Anti-beta actin mouse mAb | Cat# 12262 | Cell Signaling |
| | | Technology |

MDS Patient Samples and Institutional Review Board Approval

MDS patient samples were obtained from the left-over diagnostic specimen at the Department of Pathology, Northwestern University. The study protocol was approved by the institutional review board at Northwestern University.

Patient Database and Survival Data and Correlation with IL-6R Gene Expression

Gene expression data from 183 MDS CD34+ samples and 17 controls were obtained from GEO (GSE19429) (38) and correlated with disease subtypes and survival.

Bone Marrow Transplantation

Bone marrow transplantation was performed as described previously (6, 52, 53). Briefly, mouse total bone marrow cells were collected followed by red blood cell lysis (Invitrogen 00-4333-57). Lethally irradiated (1000 rad) recipient mice were injected retro-orbitally with approximately 2×10⁶ donor bone marrow cells. The recipient mice were then fed with water containing antibiotics for two weeks. Complete blood cell counts (Hemavet 950, Drew Scientific) and flow cytometric analysis (BD FACS Canto II) of the peripheral blood were performed at different time points after transplantation to assess chimeras and engraftment.

For the transplantation of c-Kit+ cells, splenic c-Kit+ cells from 12-month-old DKO mice and bone marrow c-Kit+ cells from 12-month-old wild-type C57/B6 mice were isolated using c-Kit (CD117) Microbeads (Miltenyi Biotech) according to manufacturer's instruction. 5-month-old CD45.1+C57/BL6 recipient mice (Charles River Laboratories) were lethally irradiated (1000 rad), 1×10⁵ c-Kit+ cells were then transplanted through retro-orbital injection.

Flow Cytometer Assay

Flow cytometer analysis was performed as previously described (52-54). Briefly, bone marrow cells were harvested from femur and tibia with phosphate buffered saline (PBS). The spleen was minced and homogenized using the frosted ends of the slides and suspended in PBS. All the cells were passed through a 40 μm cell strainer to obtain single cell suspension. The ACK lysis buffer (Thermo Fisher Scientific, A1049201) was applied to remove red blood cells when necessary. The preparation of peripheral blood was performed following previous studies (52, 55). The cells were then stained with appropriate antibodies at room temperature for 15-30 minutes, washed by PBS, and kept on ice until further analyses. Propidium iodide (PI) was added prior to the assay to exclude the dead cells. The gating strategies for hematopoietic stem/progenitor cells (HSPCs), myeloid-derived suppressor cells (MDSCs) (CD11b⁺Ly6G⁺Ly6C^{low} (granulocytic-MDSC), and CD11b⁺Ly6G⁻Ly6C^{high} (monocytic —MDSC)) have been described elsewhere (23, 52).

Treatment of Mice to Target IL-6 Signaling

Anti-horseradish peroxidase (HRP) IgG isotype control (BE0088, Bio X Cell), anti-mouse IL-6 monoclonal antibody (BE0046, Bio X Cell), and recombinant mouse gp130 Fc chimera protein (468-MG, R&D system) were diluted in InVivoPure pH 7.0 dilution buffer (IP0070, Bio X Cell) to 10 mg/100 mL and separately injected intraperitoneally into old recipient mice that have been transplanted with old DKO bone marrow cells once a week (10 mg/mouse). The treatment was started at one month post transplantation. Peripheral blood was collected retro-orbitally each month and complete blood count (CBC) of all mice were evaluated by Hemavet 950 (Drew Scientific).

For the chronic treatment, total bone marrow of 5-month-old DKO mice were collected. After RBC lysis, the cells were resuspended in PBS, 1×10 6 cells were then transplanted into 5-month-old CD45.1 lethally irradiated (1000 rad) recipient mice through retro-orbital injection. Anti-HRP IgG isotype control, anti-mouse IL-6 antibody, and gp130 Fc chimera protein were diluted as above and separately injected intraperitoneally into the above mentioned DKO transplantation mice once a week (10 mg/mouse). The treatment was started at one month post transplantation.

Enzyme-Linked Immunosorbent Assay (ELISA) Assay and Multiplex ELISA

MDS patient bone marrow aspirate samples were resuspended in 5 ml of RPMI 1640 containing 100 units/ml preservative-free sodium heparin, 100 units/ml penicillin, and 100 mg/ml streptomycin. The supernatants were collected after centrifuge. ELISA assay was performed based on the manufacturer's protocol to determine the expression levels of soluble IL-6R. Briefly, the bone marrow supernatants were diluted 50 times and incubated in antibody pre-coated 96-well plates, together with human sIL6R standard and empty controls. After mixing with HRP-conjugate, the plate was incubated at room temperature for 2 hours. After incubation, the plates were washed with washing buffer 3 times and followed by the addition of TMB substrate solution in each well and room temperature incubation for 20 minutes. The reaction was terminated by pipetting stop solution into each well and the absorption at 450 nm wavelength was measured in a spectrophotometer. All samples were performed in duplication in the assay. The same experiment was done for the detection of mouse soluble IL-6R. Mouse and human soluble interleukin 6 receptor ELISA kits were purchased from MyBioSource (MBS722764) and Thermo Scientific (BMS214), respectively.

The Mouse Magnetic Luminex Screening Kit, which detects over 40 cytokines, was purchased from R&D Systems (LXSAMSM-44). The Luminex assay was performed at the Comprehensive Metabolic Core (CMC) of Northwestern University. The mean fluorescence intensity (MFI) of each sample was calculated and analyzed. The similarity matrix and hierarchical clustering with Pearson correlation were obtained through the online tool Morpheus (software.broadinstitute. org/morpheus/).

Single-Cell RNA Sequencing

Single bone marrow mononuclear cells from aged mice were applied to the 10× Genomics platform for single-cell RNA sequencing at NUseq core facility of Northwestern University. An estimated 10,000 cells were loaded into the 10× Chromium system per sample. RNA was converted to cDNA and libraries were generated using the Chromium Single Cell 3′ v3 Kit. A Bioanalyzer was used to confirm that the main peak sizes of cDNA were generally between 450-490 bp. The prepared libraries were sent to BGI (Hongkong) with DNBseq PE 100 platform for sequencing. The sequencing data were processed (including alignment and quantification) using the Cell Ranger pipeline. The joint analysis of three single-cell datasets were performed by Seurat v4. The percentage of mitochondrial and ribosomal genes per cell were calculated and added to the metadata. The proportion of hemoglobin genes were examined to eliminate the red blood cell contamination. Filtering criteria “min. cells=3, min. features=200” was applied to filter the preliminary data. Single cells with less than 500 detected genes or more than 4000 detected genes or more than 15% reads aligned to mitochondrial genes were excluded further. For cell clustering analysis, the “IntegrateData” function in the Seurat package was used for data combination with resolution value=0.5 and 30 principal components. The UMAP algorithm was adopted to perform nonlinear dimensionality reduction analysis and cell clustering. The cluster specific marker genes were screened by calculating differentially expressed genes in each cluster compared with all remaining cells, and the cell type for each cluster was annotated and re-marked with the CellMarker database (biocc.hrbmu.edu.cn/CellMarker/index.jsp). Differentially expressed genes (DEGs) analysis were further performed between DKO vs TWT, or DKO vs TKO samples among the identical cluster. Pathway enrichments were identified according to the KEGG annotation and clarification.

Histology Staining

Mouse sternum, spleen, liver, and lung were fixed in 10% neutral-buffered formalin overnight. The samples were then embedded in paraffin and processed for hematoxylin and eosin (H&E) staining at Mouse Histology and Phenotyping Laboratory of Northwestern University. Peripheral blood or bone marrow smears were stained with May-Grunwald-Giemsa staining as previously described (6, 23).

Xenograft of MDSL Cells in NSG Mice

NOD/SCID-IL2Rg mice (NSG) mice were purchased from the Jackson Laboratory (Stock #005557). 1×10⁶ of MDS-derived cell line (MDSL) cells were retro-orbitally injected into 12-week-old sub-lethally irradiated (250 rad) female NSG mice. Ten days after transplantation, human IgG isotype control (BE0297, InVivoMab) or tocilizumab (Selleck) were administered at 8 mg/kg weekly via intraperitoneal injection. To evaluate the engraftment, peripheral blood was collected from tail vein on day 60. The peripheral blood mononuclear cells were stained with anti-human CD45 (368521, BioLegend) after red blood cell lysis (RBC lysis buffer, eBioscience) for flow cytometric analysis.

Colony-Forming Unit (CFU) Assay

Patient bone marrow derived CD34+ cells were isolated using human CD34 MicroBeads Kit (130-046-702, Miltenyi Biotec) and MACS Magnetic Separators following the manufacturer's instructions. In brief, mononuclear cells from 10 mL of total bone marrow aspirate were separated using density gradient centrifugation with Ficoll-Paque buffer (ρ=1.077 g/mL). Mononuclear cells were incubated with CD34 MicroBeads and FcR Blocking Reagent at 4° C. for 30 minutes before magnetic column separation. Collected CD34+ cells were then aliquoted to 0.3×10⁶ per vial and stored in liquid nitrogen. Frozen normal human bone marrow CD34+ cells were purchased from StemCell Technologies.

For the CFU assay, CD34+ cells were thawed in a 37° C. water bath. Cells were spun down and resuspended in IMDM medium without FBS. Cell viability was assessed with trypan blue staining and counted with Bio-Rad TC20 cell counter. 4×10³ live normal CD34+ cells were mixed with 4 mL of MethoCult Optimum medium (H4034, StemCell Technologies) supplemented with IgG or tocilizumab at the final concentration of 50 μg/mL. Given the potentially lower number of colonies generated from MDS patient CD34+ cells (56), 8×10³ live patient CD34+ cells were mixed with 4 mL of Methocult Optimum medium supplemented with human IgG isotype control or tocilizumab at the final concentration of 50 μg/mL. The cell suspension was viciously mixed by vortexing, and subsequently aliquoted at 1 mL per well in 6-well plates. The vacant wells were filled with distilled water to prevent the MethoCult from drying out. 14 days after seeding, the CFU assay was ready for evaluation. Colonies were counted under an inverted microscope (EVOS M5000, Thermo Fisher), and the colonies were identified by two independent individuals.

Testing of IL-6 Signaling

Mononuclear cells were obtained from wild-type bone marrow cells after RBC lysis (eBioscience). Cells were resuspended in serum-free RPMI1640 medium. 1×10⁶ cells then were seeded in each well of a 12-well plate. Cells were treated with anti-mouse IL-6 antibody (BE0046, InVivoMAb) or mouse gp130-Fc chimera for 1 hour under culture condition. Mouse IgG1 isotype control or control Fc fusion protein (Enzo life science) were used as negative controls for anti-mouse IL-6 antibody and mouse gp130-Fc chimera, respectively. Cells were challenged with mouse recombinant IL-6 at a final concentration of 10 ng/mL for 15 minutes before cells were harvested with RIPA buffer for western blot analyses.

Statistical Analysis

Results are expressed as mean±SEM except where otherwise indicated. The statistical analysis between two groups were performed with two-tailed unpaired Student's t test using GraphPad Prism version 8.0 software. Survival curve was compiled using Kaplan-Meier algorithms of Prism software, and the significance was assessed using the Log-rank (Mantel-Cox) test. A p<0.05 was considered statistically significant.

Results:

mDia1 and miR-146a Double Knockout Mice Progress from MDS to Acute Leukemia with Aging

DIAPH1 and MIR146A are located on chromosome 5q and are commonly deleted in patients with MDS (1, 6, 7, 27-29). Genetic abnormalities in MDS involving chromosome 5q usually show a single allele deletion. However, studies also demonstrated that many genes on the intact allele are epigenetically silenced (1, 2), which was also the case for DIAPH1 and MIR146A (FIG. 9A) (27, 30). Our recently reported study demonstrates that the constitutive mDia1/miR-146a double knockout (DKO) mice develop age-related MDS manifested as anemia, thrombocytopenia, and ineffective hematopoiesis. These mice show markedly increased inflammatory cytokines in the bone marrow and increased lethality (23). We monitored DKO mice more closely when they became moribund (12-14 months old) and dissected the mice. Compared to the double wild type (DWT) and single mDia1 or miR-146a knockout mice, the bone marrow of the majority of the moribund DKO mice (>90%) were completely replaced by monotonous blasts with marked reduction of normal bone marrow hematopoietic cells. There was also significant leukemia involvement of the spleen in that the normal splenic architecture was effaced by blasts (FIG. 1A). In addition, other organ systems were infiltrated by blasts (FIG. 1B). Marked osteosclerosis and fibrosis were also seen in the bone marrow of DKO mice (FIG. 1C). The blasts could also be observed in circulation in these mice (FIG. 1D). Consistently, the white blood cell counts in the moribund DKO mice were significantly increased (FIG. 1E). Overall, the mDia1/miR-146a double knockout mice show progression of age-related MDS to acute leukemia.

Loss of IL-6 Reverts the Leukemic Progression in DKO Mice

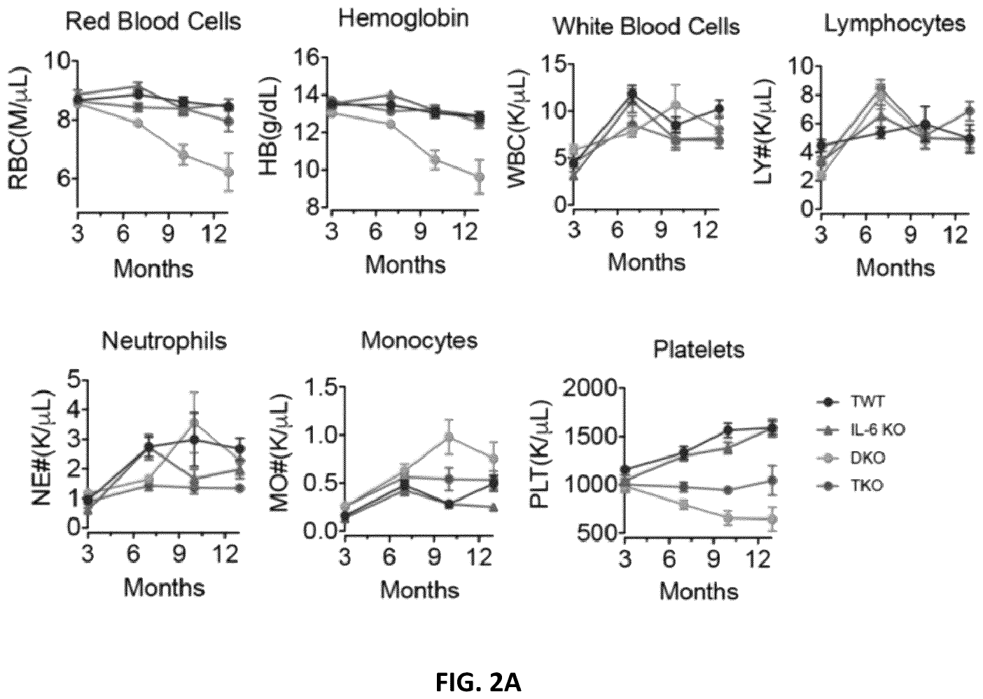

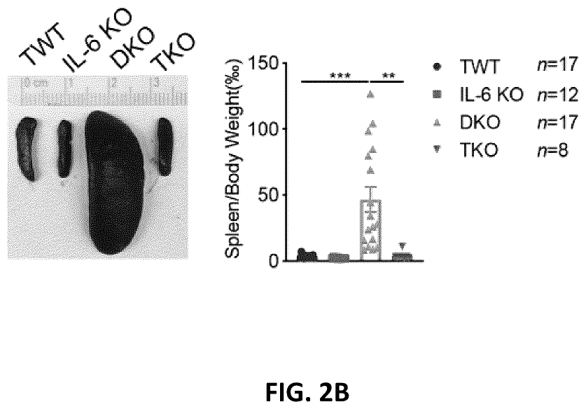

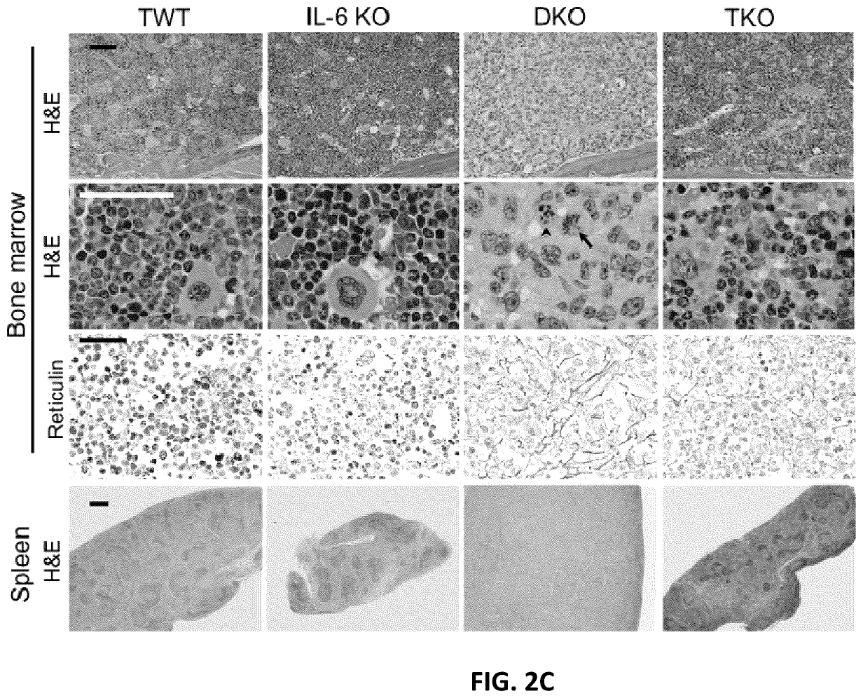

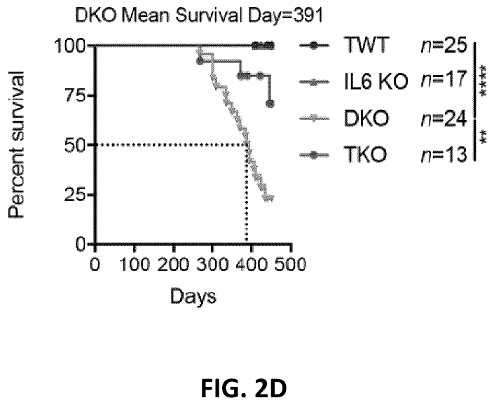

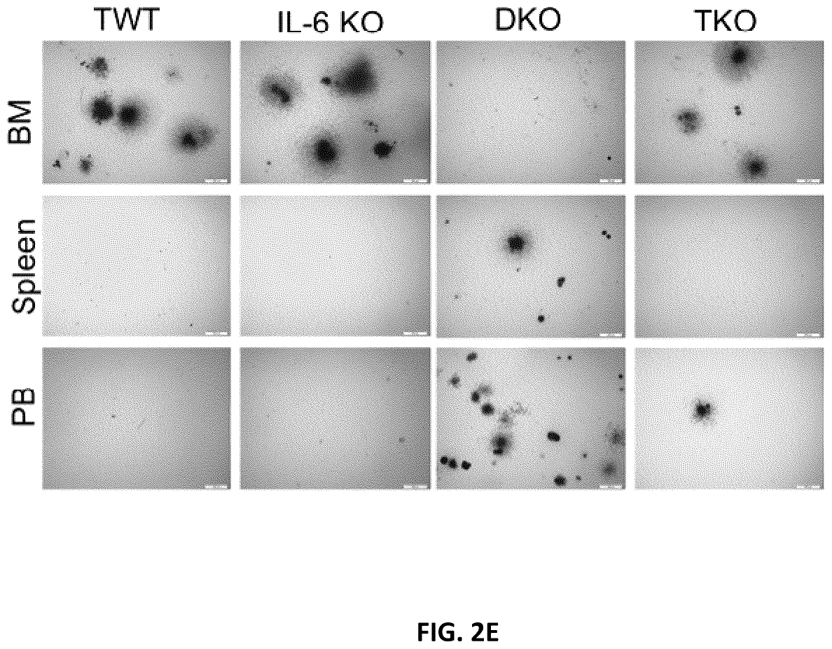

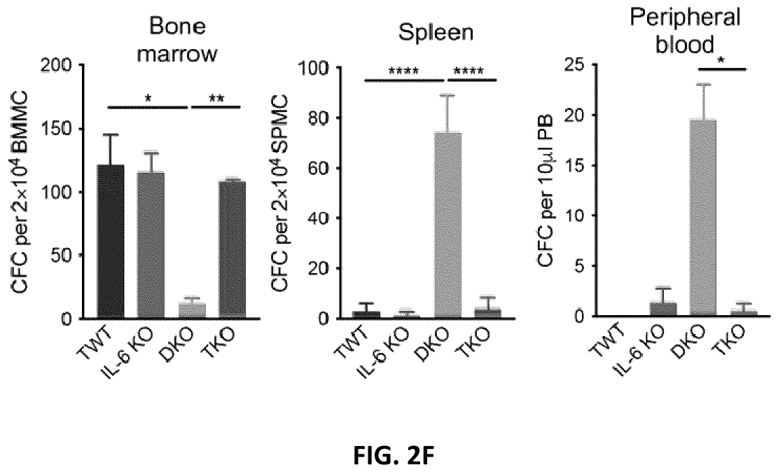

We previously demonstrated that the inflammatory bone marrow microenvironment is essential for the development of MDS in DKO mice (23). Among the inflammatory cytokines that are upregulated in the DKO mice, IL-6 shows the highest fold increase in the DKO model, indicating that it could play a major role in mediating the pathogenesis of the DKO mice. Therefore, we crossed the DKO mice with constitutive IL-6 knockout mice and generated mDia1/miR-146a/IL-6 triple knockout (TKO) mice. These mice exhibited no detectable abnormalities at steady state when they were young, including normal complete blood count (FIG. 2A). As we reported, the DKO mice started to exhibit anemia, thrombocytopenia, and monocytosis with aging. In contrast, TKO mice showed a significant reversion of these parameters (FIG. 2A). When the moribund DKO mice (12-14 months old) were sacrificed and compared to the age-matched triple wild type (TWT), IL-6 knockout, and TKO mice. The marked splenomegaly in DKO mice was reverted to the normal level in TKO mice (FIG. 2B). We next examined the histopathology of these mice. As expected, bone marrow trilineage hematopoiesis and spleen normal architecture were largely restored in TKO mice when compared to the DKO mice that showed complete replacement by blasts in these organs. The bone marrow fibrosis, as well as multi-organ leukemia infiltration, were also significantly reverted with IL-6 deficiency (FIG. 2C and FIGS. 9B to D). More important, IL-6 deficiency significantly extended the survival of the DKO mice (FIG. 2D). The replacement by blasts abolished the colony forming capacities of the bone marrow cells in DKO mice. Instead, extramedullary hematopoiesis was evident, as demonstrated by colony formation by spleen cells and circulating mononucleated cells. These changes were also reverted by the loss of IL-6 (FIGS. 2E and F). Together, these results reveal that IL-6 plays a major role in mediating MDS to leukemia progression in the DKO mice.

IL-6 is Critical for the Progression of MDS to a Myelomonocytic Leukemia in the DKO Model

We next harvested the hematopoietic tissues from these mice (12-14 months old, DKO mice at moribund) to analyze the cellular compositions and types of blasts in DKO mice and how loss of IL-6 influences the transformation process. We first analyzed peripheral blood by flow cytometry before we sacrificed the mice. Consistent with the complete blood count, the DKO mice contained increased percentages of granulocytic (CD11b+, Ly6G+, and Ly6C−) and monocytic (CD11b+, Ly6G−, and Ly6C+) populations. Lymphocytes were significantly reduced (FIG. 3A). We previously reported that DKO mice contained an increased number of myeloid derived suppressor cells (MDSCs) that include both granulocytic-MDSC (CD11b⁺Ly6G⁺Ly6C^{low}, same as granulocytes in this model) and monocytic-MDSC (CD11b⁺Ly6G⁺Ly6C^{high}) (23). Given this information and the presence of blasts in the peripheral blood of the DKO mice, we analyzed the CD11b+ myeloid populations. Among these cells, the Ly6C monocytic population was further divided into Ly6C high, medium, and low cells (FIG. 3B). Compared to the blood mononuclear cells from TWT and single knockout mice, those from the DKO mice contained a particularly high percentage of CD11b+/Ly6C^{low} cells, which are likely to be within the blast population (FIG. 3C). Indeed, we found that the CD11b⁺/Ly6C^{low} cells in the DKO mice were c-Kit+. Similarly, loss of IL-6 reverted this phenotype (FIG. 3D).

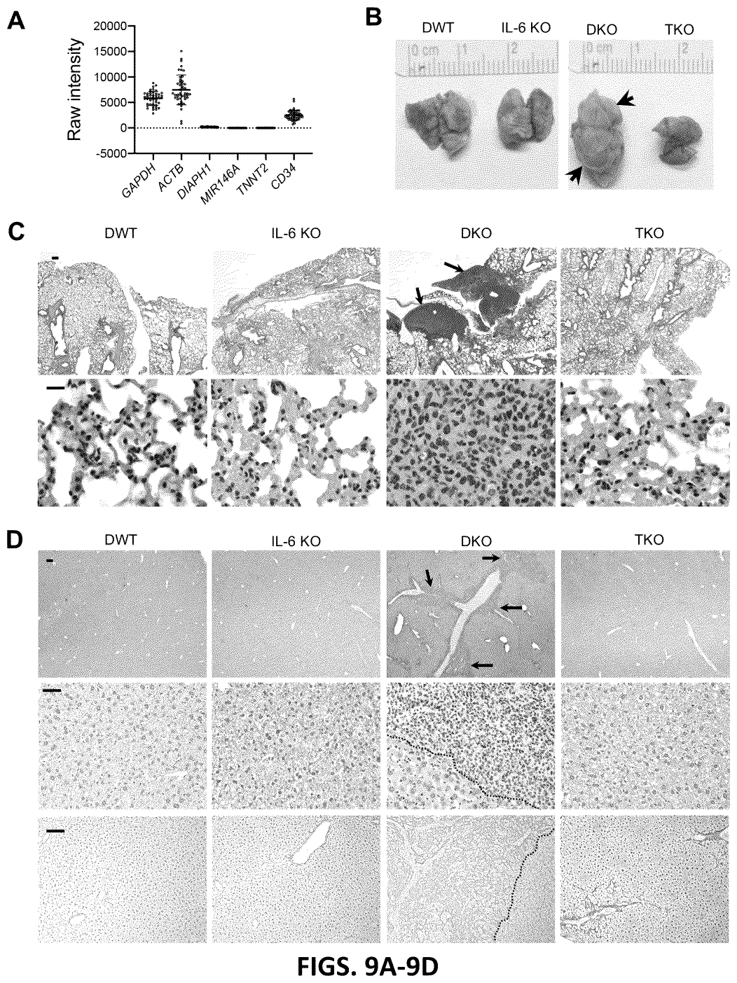

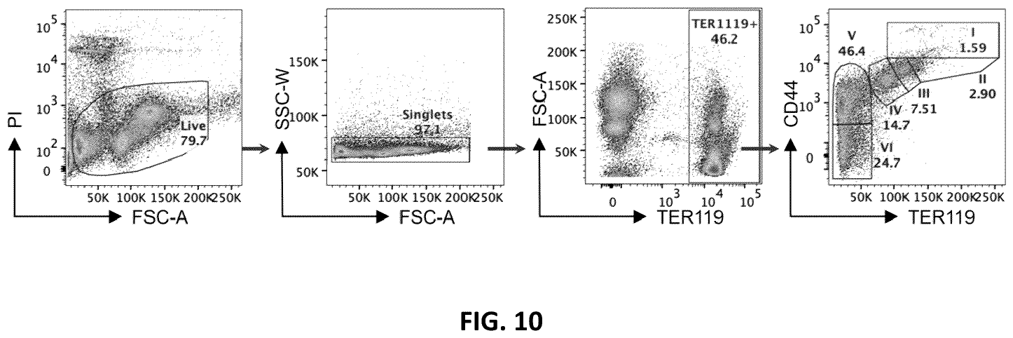

We next analyzed the bone marrow and spleens of these mice. Consistent with the findings in the peripheral blood, the overall CD11b+/Ly6G− monocytic populations, including Ly6C high, medium, and low subpopulations, were significantly increased in the bone marrow of DKO mice. These monocytic cells, as well as Ly6G+ granulocytic population that together comprised MDSC, were dramatically increased in the spleen, which is consistent with the marked splenomegaly in DKO mice (FIG. 3E). These cells were also relatively larger, which is consistent with their shift to immaturity when compared to wild type control cells (FIG. 3F). Again, loss of IL-6 nearly completely reverted these phenotypes (FIGS. 3E and F). Flow cytometry assays also revealed a dramatic reduction in cells at all stages of terminal erythropoiesis in the bone marrow of DKO mice. This was associated with marked extramedullary erythropoiesis in the enlarged spleen in these mice. Loss of IL-6 largely reverted these phenotypes in erythropoiesis as well (FIG. 3G and FIG. 10). Overall, these results reveal that the DKO mouse represents a model of MDS transformation to acute myelomonocytic leukemia. The blast population in the leukemia phase is predominantly monocytic c-Kit+ cells.

Single Cell RNA Sequencing and Cytokine Analyses of DKO and TKO Models

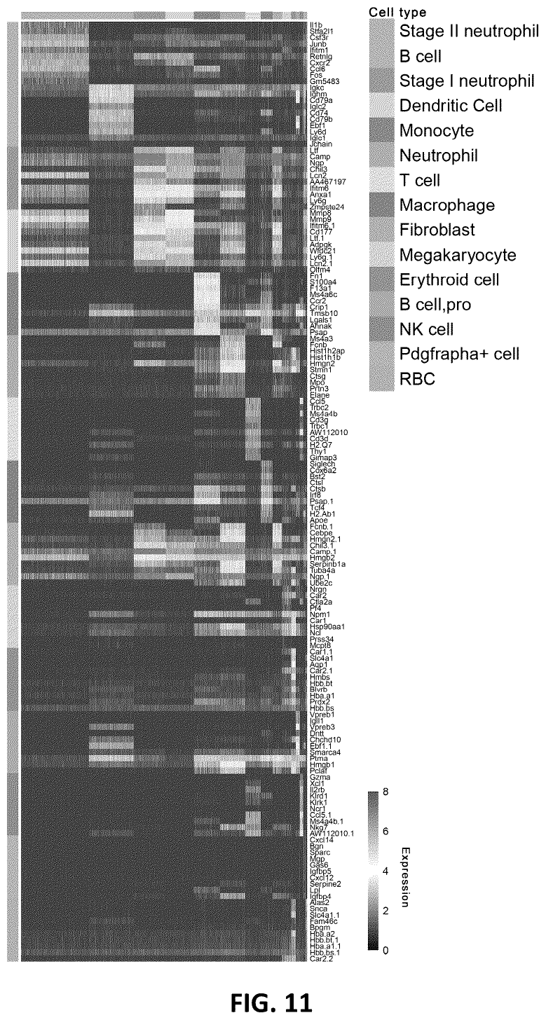

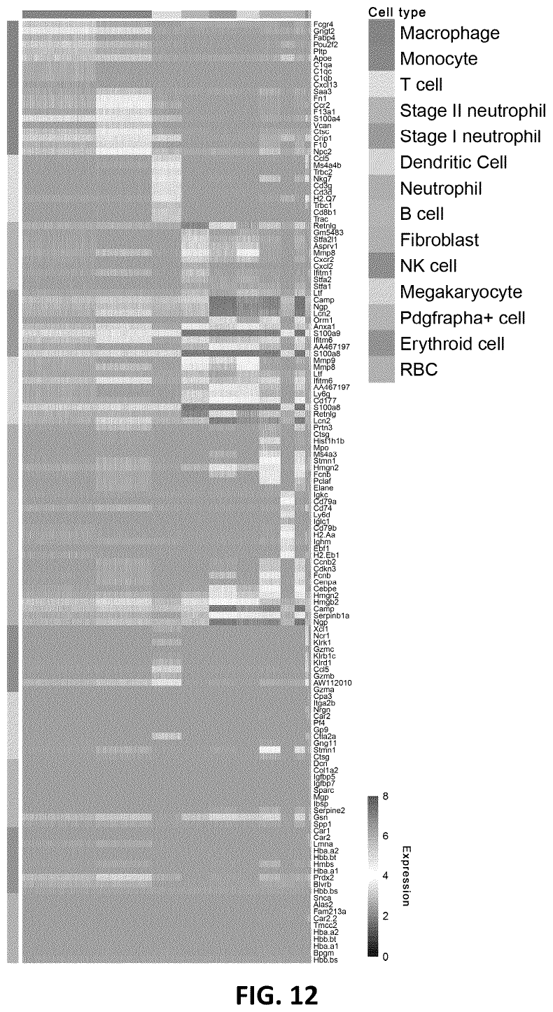

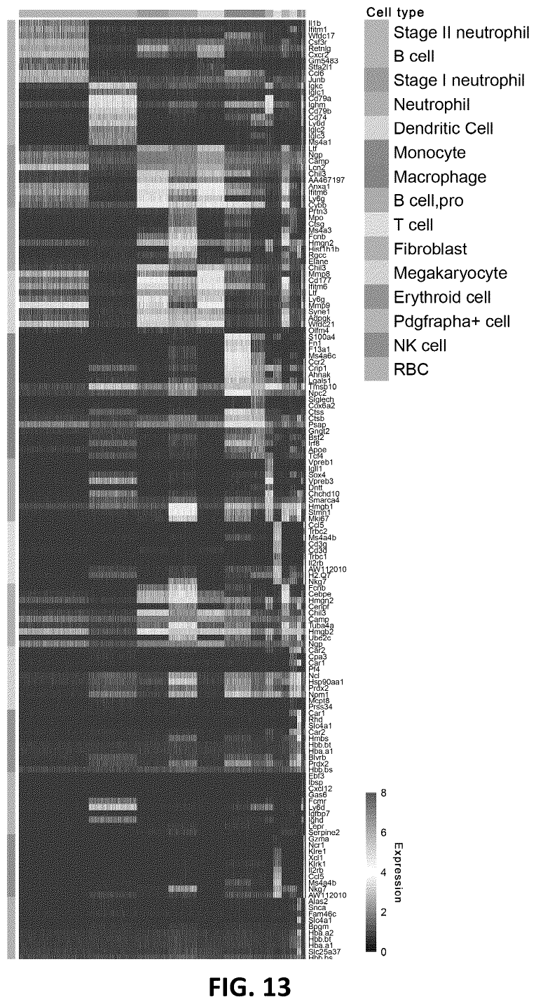

To investigate the leukemic transformation in DKO mice at the single cell level, we sequenced 8423, 10866 and 9013 mononuclear cells from the bone marrow of 12-14 months old TWT, DKO and TKO mice, respectively. TWT and TKO individual cell has the comparable median gene number (TWT, 1332 vs TKO, 1384) and median unique molecular identifier (UMI) transcripts (TWT, 4363 vs TKO, 4591). In contrast, the DKO single cell has higher median gene number (1744) and UMI transcripts (6596). The most significant marker gene expression profiles in each cluster exhibited similarities between TWT and TKO mice, whereas DKO cells had a more unique pattern (FIGS. 11-13).

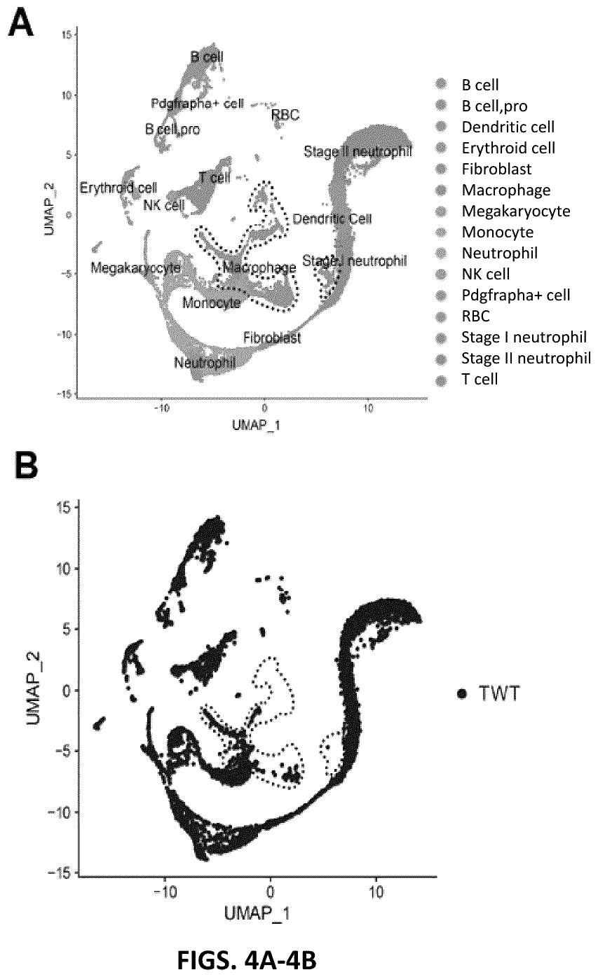

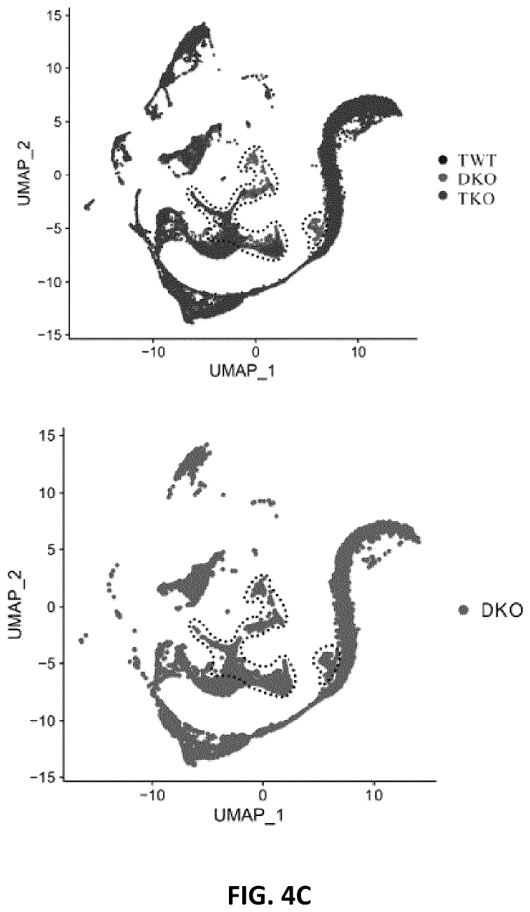

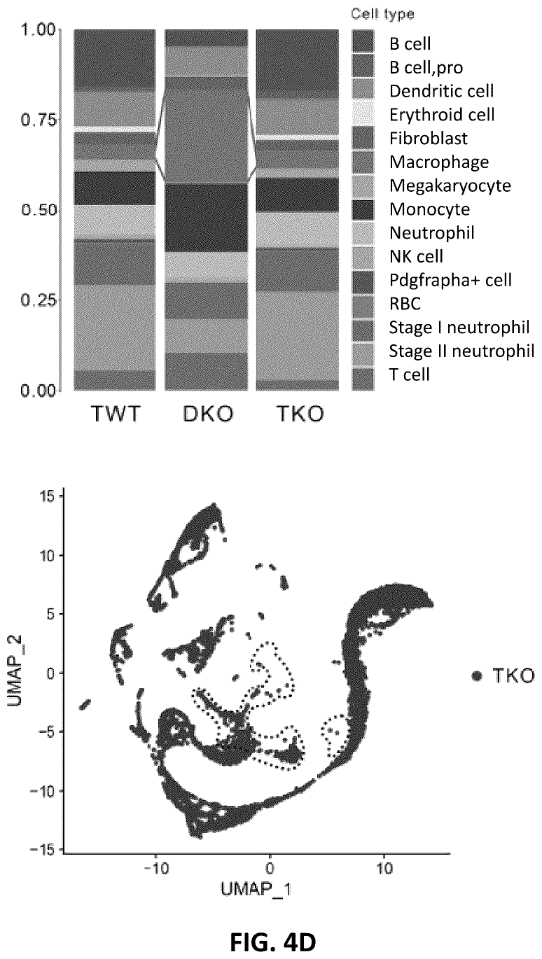

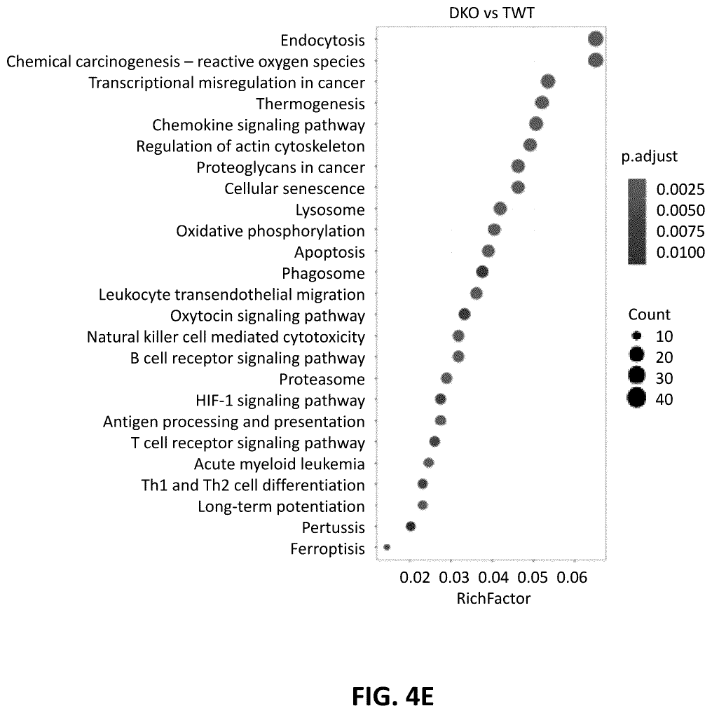

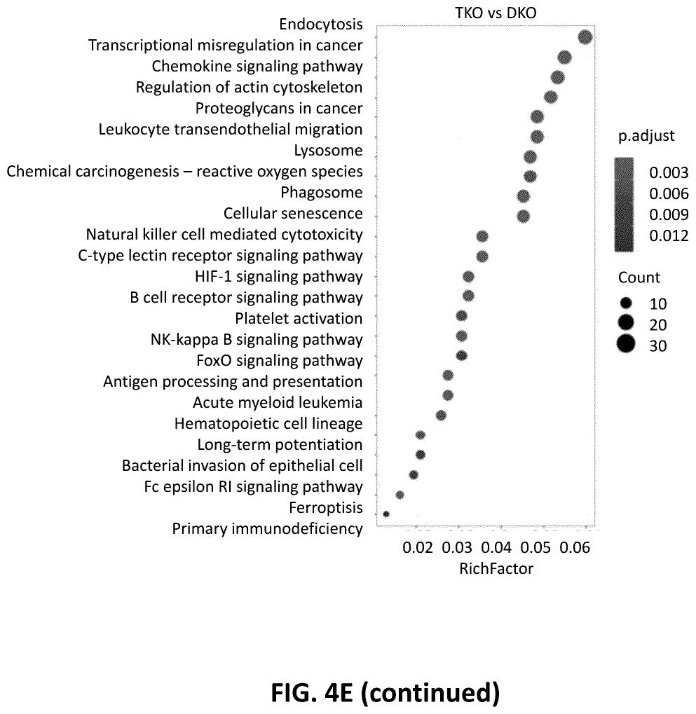

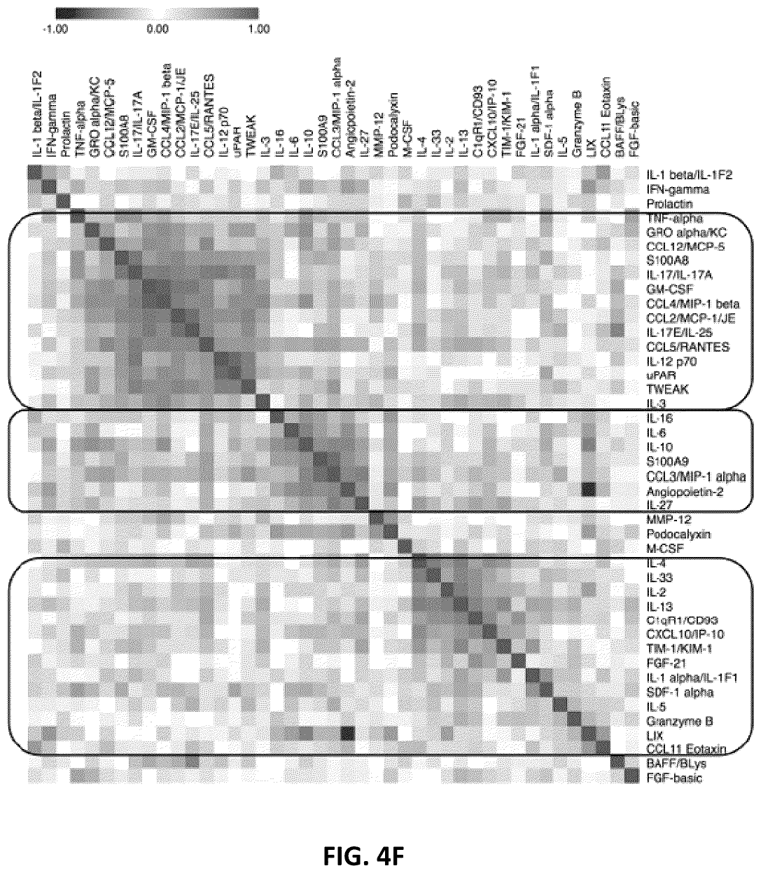

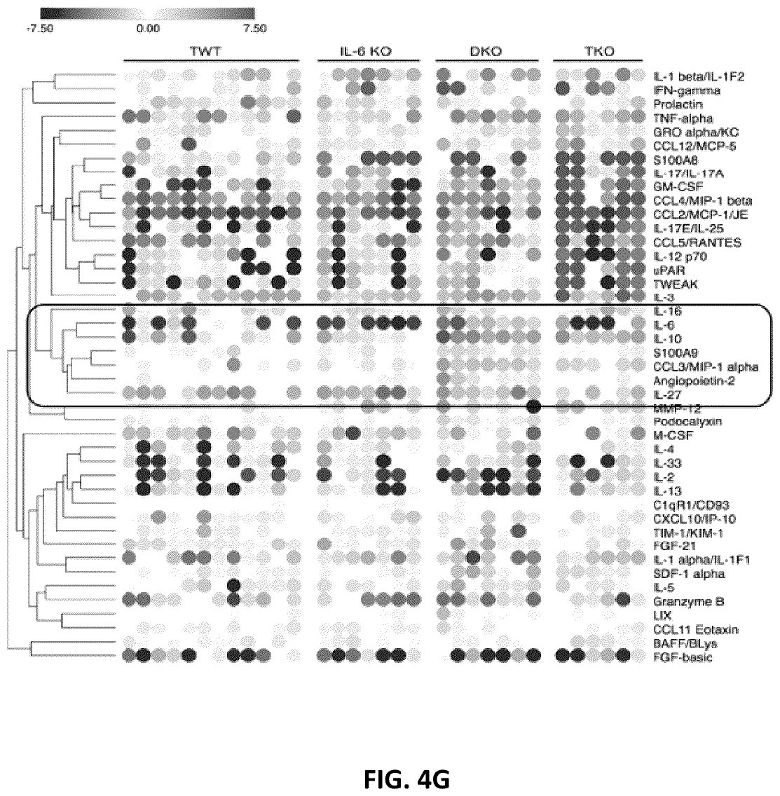

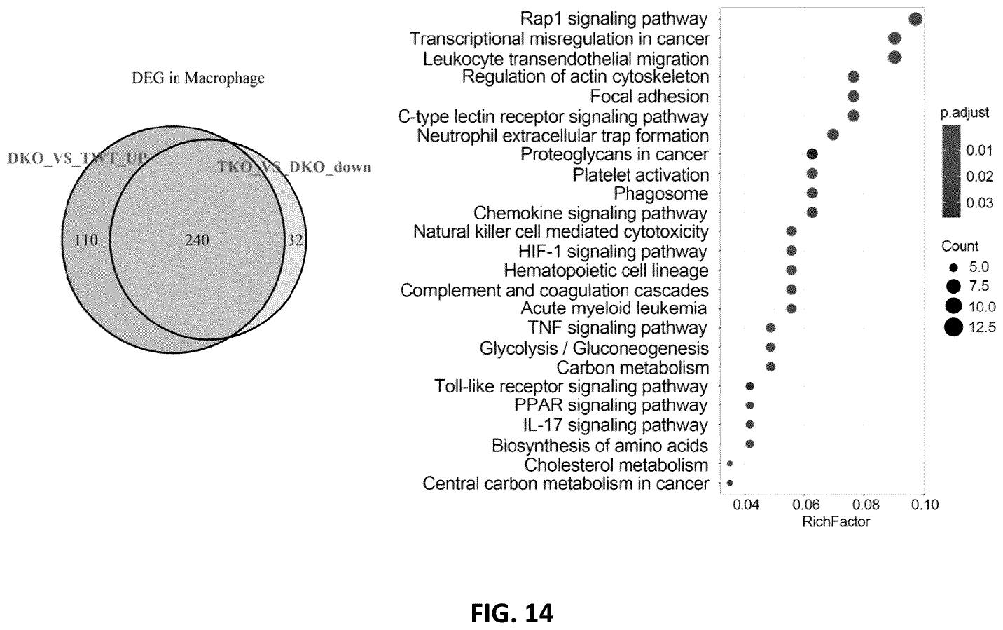

Integrated analysis identified altered cell populations in the bone marrow of DKO mice, which were corrected by IL-6 deficiency in TKO mice (FIGS. 4A and B). We captured a significant accumulation of cells expressing monocyte, macrophage, and T cell markers in DKO bone marrow, which were at low levels in TWT and TKO bone marrow (FIGS. 4A-D). These findings are consistent with the flow cytometry data in which the immature monocytic cells represent the blast population (FIG. 3). Further analyses examined the differential expressed genes (DEGs) among all clusters in different genotypes, and the KEGG pathway enrichment analysis uncovered a significant enrichment of DEGs involved in various signaling pathways in cancers (FIG. 4E). Using DEGs in clusters expressing macrophage markers as an example, KEGG pathway analysis of the altered genes identified many unique pathways that are upregulated in DKO cells and reverted in TKO cells (FIG. 14). IL-6 is one of the highly upregulated pro-inflammatory cytokines in DKO mice in our previous report (23). To comprehensively reveal the changes of various inflammatory cytokines upon IL-6 depletion, we analyzed the serum from the old mice (12-14 months) and performed a multiplex ELISA assay. A cytokine pairwise similarity assay identified three major clusters among cytokine expression pattern in all 4 groups (FIG. 4F). A non-biased hierarchically clustered heatmap analysis further confirmed clusters of cytokines overproduced in DKO mice, including IL-6. While IL-6 level was markedly reduced in the TKO serum, the levels of other inflammatory cytokines remained unexpectedly high (FIG. 4G). These data indicate that IL-6 is pivotal in driving the progression from MDS to AML in this mouse model.

The DKO Leukemia Model is Transplantable

We previously reported that wild-type recipient mice transplanted with DKO bone marrow cells developed MDS with similar age-related hematologic phenotypes in DKO mice. These mice succumbed to the disease with aging (23). We performed similar transplantation experiments with the addition of TKO and IL-6 KO groups (FIG. 5A). Indeed, loss of IL-6 significantly reverted the age-related lethality in this model (FIG. 5B). In these assays, the donor cells were from younger mice since bone marrow cells in leukemia phase DKO mice were difficult to obtain due to fibrosis. To model the direct leukemia cell engraftment in the recipient mice, we purified spleen cells from the moribund DKO mice and their age-matched counterparts in other groups. The spleen cells were then transplanted into lethally irradiated young wild type mice (FIG. 5A). Consistent with the data that the majority of colony-forming hematopoietic stem and progenitor cells were in the bone marrow (FIG. 2E), spleen cells from TWT, IL-6 KO, and TKO mice were ineffective in engrafting the recipient mice. However, those that survived stayed alive. In contrast, all mice transplanted with splenic cells from leukemic DKO donor mice survived the initial post-transplantation stage but rapidly died shortly after (FIGS. 5C and D). The moribund recipient mice transplanted with leukemic DKO splenic cells showed marked leukocytosis (including all myeloid lineages), anemia, and thrombocytopenia (FIG. 5E). Many circulating blasts were readily identified and expressed c-Kit (FIGS. 5F and G). We further analyzed the c-Kit+ cells using flow cytometry and found most of these cells were CD11b positive but negative for both Ly6G and Ly6C, demonstrating their nature of immaturity (FIG. 5H).

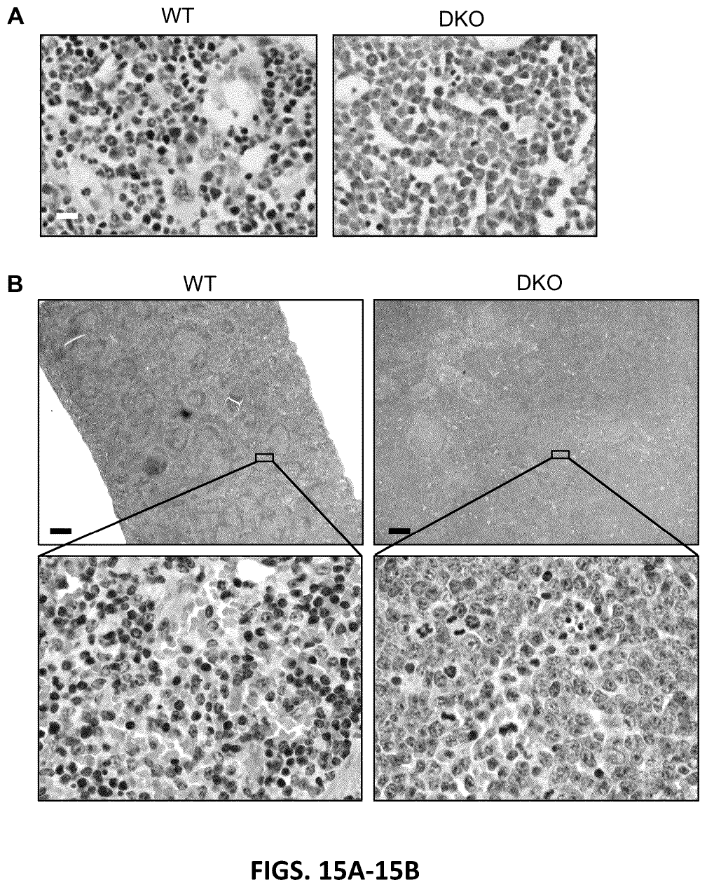

To further demonstrate that the transplantable leukemia is derived from the blast population, we purified c-Kit+ cells from the spleens of 12-month-old DKO mice and transplanted them into 5-month-old lethally irradiated recipient mice. For the control group, we used bone marrow c-Kit+ cells from age-matched wild type littermates since there are few c-Kit+ cells in the spleen of these mice. As expected, the recipient mice transplanted with the DKO splenic c-Kit+ cells developed leukemia within 5 months after transplantation with similar phenotypes as the moribund DKO mice (FIGS. 15A and B).

IL-6 Receptor and Soluble IL-6 Receptor are Increased in the Bone Marrow of Patients with High Risk MDS

The pivotal roles of IL-6 in mediating MDS to AML progression in the DKO mouse model prompted us to investigate IL-6 signaling in human patients with MDS. IL-6 is well known to be upregulated in MDS (31-34). Through its classic pathway, IL-6 binds to cell surface IL-6 receptor (IL-6R) and gp130 to trigger the downstream signaling. This pathway is believed to be involved in the protective and regenerative functions of IL-6. On the other hand, the pro-inflammatory functions of IL-6 are mostly mediated through its trans-signaling pathway in which IL-6 and soluble IL-6R (sIL-6R) complex bind to the ubiquitously expressed gp130 in many different cell types (35-37). The expression levels of IL-6R in MDS are unclear. We first analyzed IL-6R levels in CD34+ hematopoietic progenitor cells in a published dataset (38). We found that cells from low risk MDS patients, including MDS with single lineage dysplasia and MDS with ring sideroblasts, did not show differences in IL-6R mRNA expression compared to those from healthy control patients. In contrast, IL-6R was significantly upregulated in cells from patients with high risk MDS, including MDS with excess blasts 1 and 2 (FIG. 6A). Patients with high IL-6R expression were also associated with lower survival rate compared to those with low level expression (FIG. 6B). Consistent with these data, immunohistochemical stains in the bone marrow revealed a significant upregulation of IL-6R in most of the bone marrow cells in high risk MDS compared to control group individuals and patients with low risk MDS (FIG. 6C).

Unlike the relatively restricted expression of IL-6R, gp130 is expressed on most cell types that could mediate IL-6 signaling when IL-6 binds to a soluble form of IL-6R. Therefore, we analyzed soluble IL-6R (sIL-6R) in patients with various subtypes of MDS. Indeed, we found a heterogeneous but significantly increased level of bone marrow sIL-6R in MDS compared to the control group. Interestingly, the increase in sIL-6R was not observed in the serum in MDS patients (FIG. 6D). Similar to the cell surface IL-6R expression patterns, the level of sIL-6R was significantly upregulated in the bone marrow of patients with high risk MDS, but not in low-risk subtypes (FIG. 6E). We next determined whether we could observe the same phenotypes in our mouse models. Like patient serum, there were no statistically significant differences in sIL-6R levels among various groups of mice in blood (FIG. 6F). We could not obtain an adequate amount of bone marrow aspirate in the DKO mice due to significant fibrosis. Therefore, we performed flow cytometric assays on different lineages of hematopoietic cells in the bone marrow and spleen of these mice. As expected, surface IL-6R levels were significantly increased in DKO mice (FIG. 6G). The level of surface IL-6R was especially high in the bone marrow erythroid cells in DKO mice, which was unexpected since the majority of the IL-6R expressing cells were monocytic cells in wild-type mice. These upregulations of surface IL-6R were normalized in TKO mice. Together, these data indicate an important role of IL-6 signaling in MDS progression to AML in both human MDS and DKO mouse models.

Targeting IL-6 Signaling Ameliorates MDS to AML Progression in DKO Model

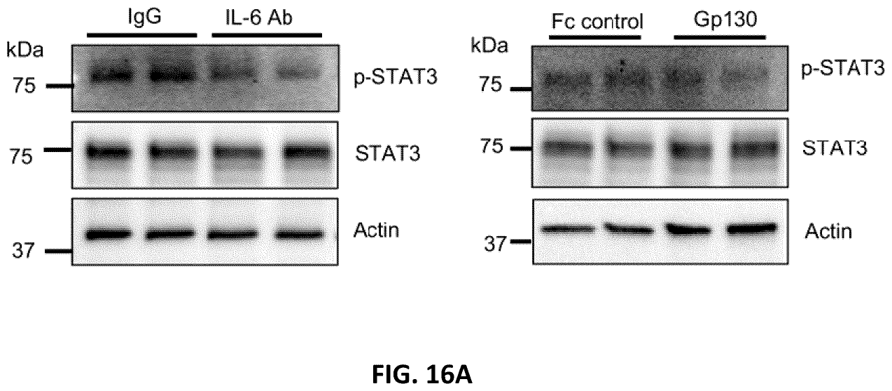

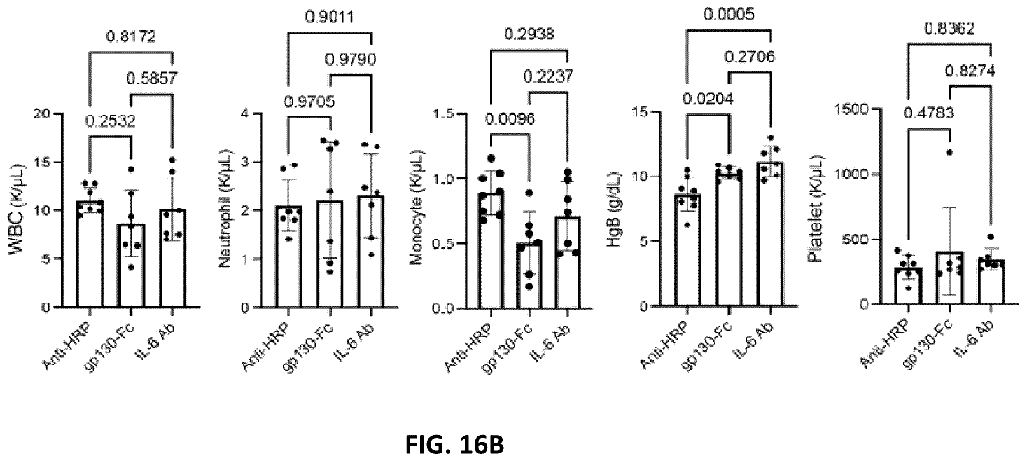

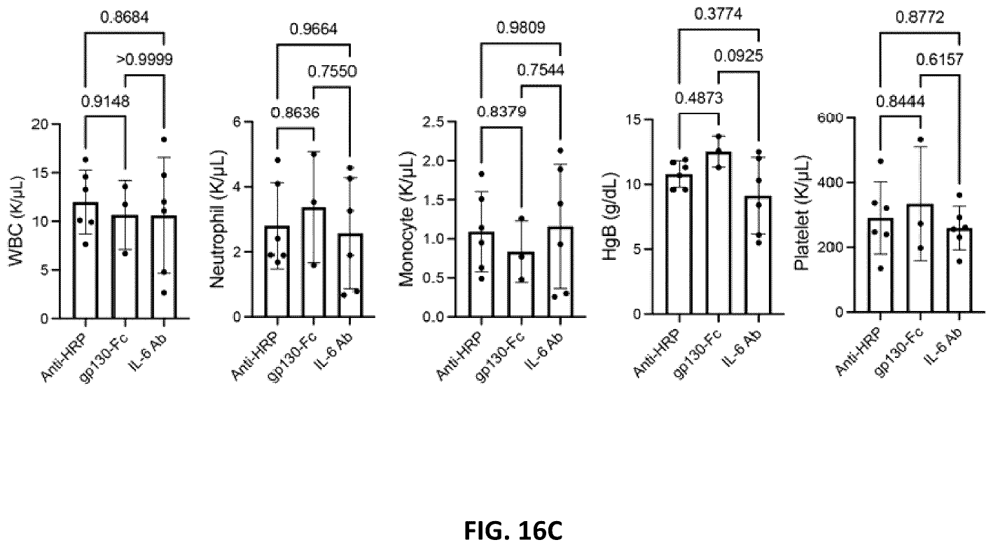

Given the critical roles of IL-6 signaling in the progression of MDS to AML, we reasoned that inhibition of this pathway will ameliorate the phenotypes of the DKO mice. To test this, we purified total bone marrow cells from 12-month-old DKO mice that were still in the MDS stage and transplanted them into 12-month-old lethally irradiated recipient mice (FIG. 7A). Through this strategy, we will be able to model the MDS to AML progression in a relatively short period with efficient initial engraftment compared to the use of AML stage splenic cells as donors (FIG. 5). We treated these mice with anti-mouse IL-6 antibodies or recombinant mouse gp130 Fc chimera proteins and compared with control group mice treated with anti-horseradish peroxidase (HRP) mouse IgG isotype. We first confirmed the efficacies of the IL-6 antibody and gp130 Fc chimera protein in downregulating IL-6 signaling in mouse bone marrow cells (FIG. 16A). The mice in the control group developed lethal AML rapidly after transplantation within two months. Treatment of the mice with anti-IL-6 antibody, and specifically gp130 Fc, significantly extended the survival (FIG. 7B). We sacrificed these mice when the control group mice became moribund (40 days post-transplant). The mice treated with anti-HRP IgG isotype showed pancytopenia and replacement of normal hematopoiesis by blasts in the bone marrow. The normal architecture of the spleen was also effaced by blasts. The liver showed significant blast infiltration. These phenotypes were ameliorated with the treatment of IL-6 antibody, and particularly gp130 Fc in this model (FIGS. 7C and D).

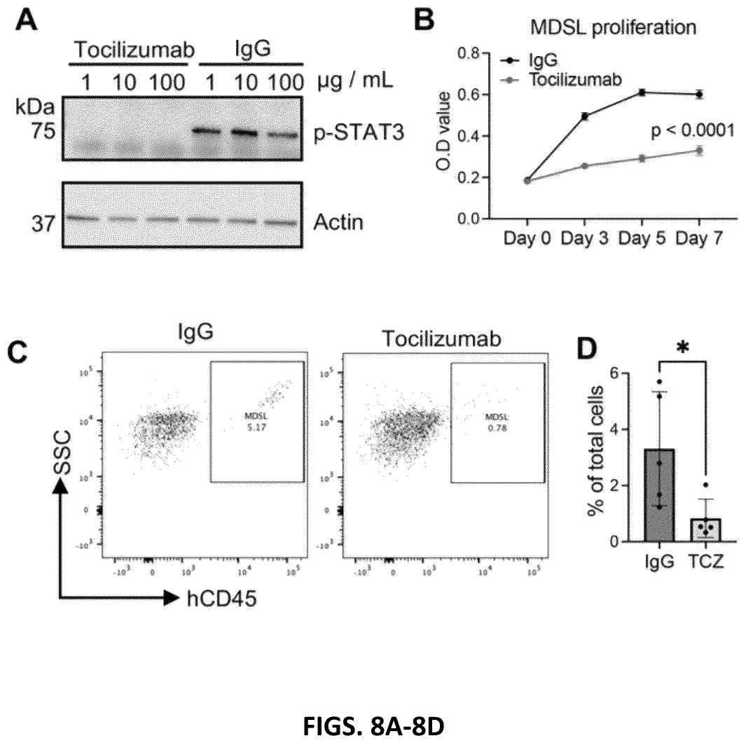

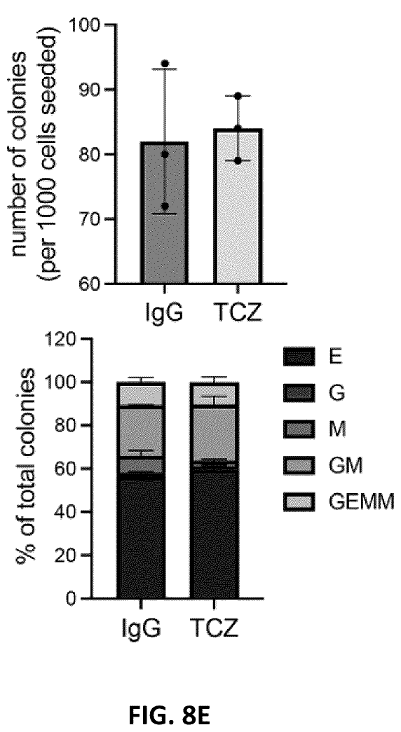

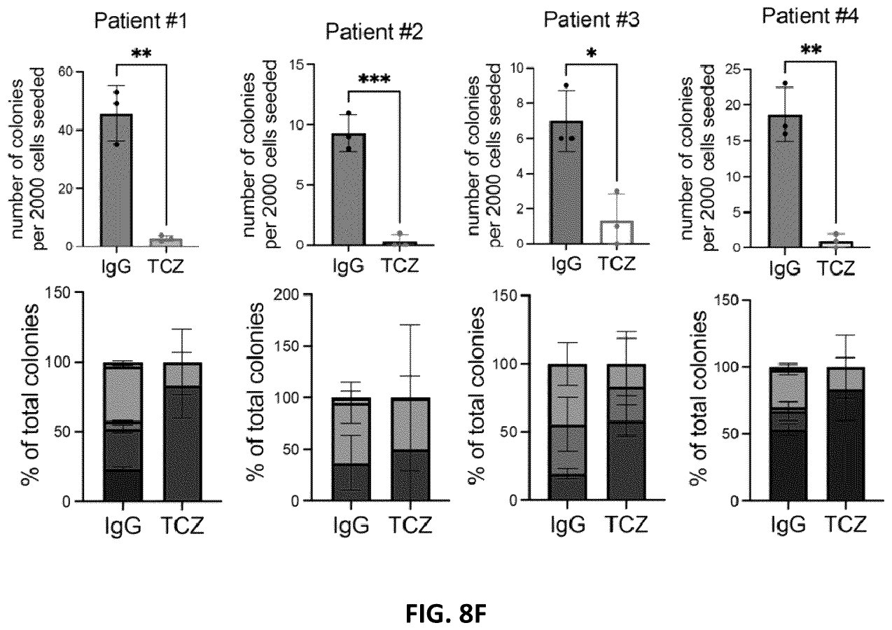

To determine whether inhibition of IL-6 signaling at a younger age could prevent the development of MDS in the DKO mice, we treated 5-month-old wild type recipient mice transplanted with the bone marrow cells from 5-month-old DKO mice chronically with anti-IL-6 antibody or gp130 Fc chimera protein. We found partial reversion of the MDS phenotypes at 1 month post treatment but loss of efficacies when the mice were tested 4 months post treatment (FIGS. 16B and 8C). These results further support the critical role of increased IL-6 signaling during MDS to AML progression, but not at the early stage of MDS development.

Anti-IL-6R Antibody Reduces Cell Proliferation and Clonogenicity in MDS Patient Cells

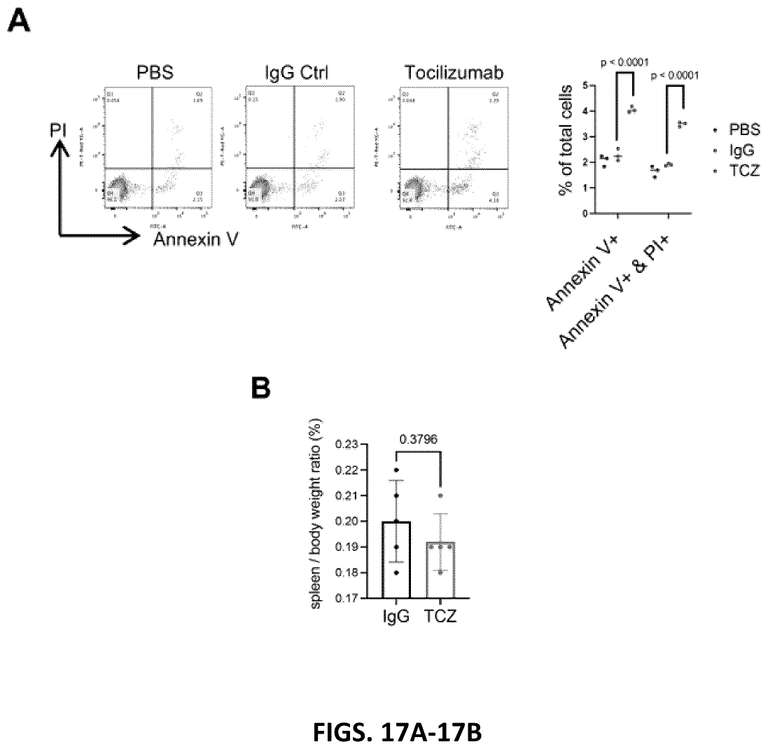

To further investigate the role of IL-6 signaling in the progression of MDS to AML in patients, we first used MDS-derived cell line (MDSL) cells. These cells were originally derived from a MDS patient and maintain the potential to engraft immunocompromised NOD/SCID-IL2Rg mice (NSG) (39). We treated MDSL cells with tocilizumab, which is a monoclonal antibody against IL-6R and clinically used to treat rheumatoid arthritis. This led to a significant reduction of p-STAT3 in vitro (FIG. 8A), decreased cell proliferation (FIG. 8B), and partial induction of cell death (FIG. 17A). We then treated the NSG mice transplanted with MDSL cells with tocilizumab and found significantly reduced MDSL engraftment (FIGS. 8C and D). Tocilizumab also reduced the spleen weight that was increased due to MDSL infiltration in these mice (FIG. 17B).

To evaluate the effects of tocilizumab in primary cells from MDS patients, we purified bone marrow CD34+ blast population that contains hematopoietic stem and progenitor cells from 4 patients with high risk MDS. These patients harbor cytogenetic abnormalities and somatic mutations that are commonly seen in myeloid neoplasms (Table 2). Tocilizumab did not affect colony expansion or composition in normal bone marrow CD34+ cells in an in vitro colony assay (FIG. 8E). The colonies derived from MDS CD34+ cells expanded less robustly compared to their normal counterparts and were myeloid skewed with less erythroid colonies. In contrast to the normal cells, the colony numbers were markedly reduced when tocilizumab was applied in MDS derived CD34+ cells (FIG. 8F). Together, these results reveal that anti-IL-6R antibody is effective in reducing cell proliferation and colony formation in MDS patient cells.

TABLE 2
Clinical information for patients with high risk MDS

| | Patient #1 | Patient #2 | Patient #3 | Patient #4 |
|---|---|---|---|---|
| Age (year) | 80 | 74 | 66 | 69 |
| Gender | Female | Male | Male | Female |
| Diagnosis | Myelodysplastic | High grade myeloid | Myelodysplastic | Myelodysplastic |
| | syndrome with | neoplasm | syndrome with | syndrome with |
| | multilineage | | excess blasts-2 | excess blasts-2 |
| | dysplasia | | (MDS-EB-2) | (MDS-EB-2) |
| | (MDS-MLD) | | | |
| Bone | 2% | 15-20% | 10% | 15% |
| marrow | | | | |
| blasts | | | | |
| Next | ASXL1, PHF6, and | ASXL1, SRSF2, | Two TP53 mutations | IDH2 and BCOR |
| generation | TET2 mutations | SETBP1, and KRAS | | mutations |
| sequencing | | mutations | | |
| Karyotype | 46, XX[20] | 47, XY, +13, +17, | 48~50, XY, add(3)(q12), | 47, XX, +4[2] |
| | | del(17)(p13p11.2), | der(5)t(5; 15)(q15; | |
| | | del(12)(p13p12), | q11.2), +6, add(6)(q23), | |
| | | dic(17; 20) | del(7)(q22q36), +8, | |
| | | (p11.2; q11.2)[cp6] | del(9)(p24p13), | |
| | | | der(11)add(11)(p11.1)del(11)(q13q21), | |
| | | | −13, −15, −19. | |
| | | | +3~6mar[cp18] | |

Discussion:

Approximately 20-30% of patients with MDS progress to AML (40). The prognosis after AML progression is dismal with limited treatment options. This unmet medical need necessitates the development of novel therapies to block or delay the progression (14, 41). In this study, the mDia1/miR-146a double knockout mice represent one of the first models that phenocopy MDS to AML progression induced by the aging bone marrow inflammatory microenvironment. Through this model, we demonstrate that IL-6 and its signaling pathway play a pivotal role in mediating the disease progression.

In our previous study, we found severe anemia in DKO mice that could cause mortality. However, the leukemic transformation in DKO mice occurred within a relative short period prior to death, which led to a missed diagnosis of leukemia in the moribund mice in the previous study. In the current work, we examined the bone marrow and blood more closely, especially in the moribund mice. Leukemia transformation was discovered in most of the moribund mice we investigated. In this respect, the phenotypes of the moribund DKO mice closely mimic patients who are transformed from high risk MDS to AML. The prognosis in these transformed patients is also dismal and death often occurs within a year of secondary AML diagnosis (2, 42). This period reflects a rapid lethality in the DKO model when the animals are transformed.

The dramatic rescue effects of IL-6 deficiency on AML progression in the DKO mice is unexpected since multiple inflammatory cytokines are upregulated. Moreover, loss of IL-6 in these mice does not significantly reduce the levels of other cytokines, which indicates that IL-6 could function downstream or independent of most of these cytokines. Indeed, studies have shown that IL-1b induces IL-6 through a phosphatidylinositol 3-kinase dependent pathway (43). IL-1b is a critical mediator of the inflammatory response and is downstream of NLRP3 inflammasome, which is reported to function as a driver of MDS phenotype (44). The level of IL-1b is unchanged in the TKO mice compared to their DKO counterparts. However, the roles of IL-1b and NLRP3 in the pathogenesis of the DKO model remain to be determined. Furthermore, it is likely that other cytokines, especially tumor necrosis factor alpha which is also highly upregulated in the DKO mice (23), could also play critical roles in parallel with IL-6.

IL-6 is known to be oversecreted in many hematological malignancies (45), including MDS (31, 32). While the pathophysiology of IL-6 signaling in several lymphoid neoplasms were well studied (46-49), the roles of IL-6 in MDS remain observational. A phase 2, randomized, double-blind multicenter study comparing anti-IL-6 effects with placebo in anemic patients with international prognostic scoring system low or intermediate-1-risk MDS showed no reduction in RBC transfusions in transfusion-dependent patients (50). However, the study was done without the knowledge of IL-6 levels in the patients and high risk MDS patients were not included. It is also unclear whether the serum or bone marrow levels of IL-6 and IL-6R are increased in different subtypes of MDS.

Consistent with this clinical study, we found that IL-6 plays less important roles in the early stage of MDS in our DKO model. This conclusion is based on the following observations. First, the MDS phenotypes remain in the TKO mice. For example, TKO mice continued to have thrombocytopenia, reduced survival, and increased immature monocytic cells compared to their age-matched wild-type counterparts. Second, chronic treatment of young DKO mice with anti-IL-6 agents failed to revert MDS phenotypes albeit these mice may develop resistance to IL-6 antibody. Nevertheless, a definitive answer to the question of the role of IL-6 in MDS initiation would require a genetic model in which IL-6 is deleted hematopoietic specifically and temporally controlled in the DKO mice. It is noted that the efficacies of the anti-IL-6 treatment in the DKO mice are less dramatic compared to the genetic depletion of IL-6, which could be due to the less effective downregulation of IL-6 signaling by these agents. In this respect, genetic approaches will be more important in future studies. It should also be noted that gp130 may also influence other cytokines to show a better efficacy than anti-IL-6 antibody in this model.

In the current study, while an increased level in the serum of patients with MDS was not observed, sIL-6R was highly upregulated in the bone marrow aspirate solution of MDS patients, which highlights the significance of the inflammatory bone marrow microenvironment in the pathogenesis of MDS (12, 51). Notably, sIL-6R levels in the bone marrow did not show differences between the control group and low risk MDS. The difference became significant in the high risk MDS groups, which further underlies the roles of IL-6 signaling in the progression of MDS to AML. Consistently, treatment of old DKO mice with anti-IL-6 agents significantly ameliorated

AML progression and extended their survival. More importantly, tocilizumab markedly reduced the engraftment of a human MDS cell line in a xenograft model. Tocilizumab also significantly reduced clonogenicity in primary CD34+ cells from high risk MDS patients. These studies indicate that therapeutic management of patients with high risk MDS, especially those with high bone marrow levels of sIL-6R, through the intervention of the IL-6 signaling pathway, such as using tocilizumab, could be beneficial by reducing the progression to AML.

### REFERENCES

1. Lindsley R C, and Ebert B L. Molecular pathophysiology of myelodysplastic syndromes. Annu Rev Pathol. 2013; 8:21-47.
2. Gangat N, Patnaik M M, and Tefferi A. Myelodysplastic syndromes: Contemporary review and how we treat. Am J Hematol. 2016; 91(1):76-89.
3. Haferlach T, Nagata Y, Grossmann V, Okuno Y, Bacher U, Nagae G, et al. Landscape of genetic lesions in 944 patients with myelodysplastic syndromes. Leukemia. 2014; 28(2):241-7.
4. Link D C, and Walter M J. ‘CHIP’ping away at clonal hematopoiesis. Leukemia. 2016; 30(8):1633-5.
5. Steensma D P, Bejar R, Jaiswal S, Lindsley R C, Sekeres M A, Hasserjian R P, et al. Clonal hematopoiesis of indeterminate potential and its distinction from myelodysplastic syndromes. Blood. 2015; 126(1):9-16.
6. Keerthivasan G, Mei Y, Zhao B, Zhang L, Harris C E, Gao J, et al. Aberrant overexpression of CD14 on granulocytes sensitizes the innate immune response in mDia1 heterozygous del(5q) MDS. Blood. 2014; 124(5):780-90.
7 Starczynowski D T. Errant innate immune signaling in del(5q) MDS. Blood. 2014; 124(5):669-71.
8. Varney M E, Niederkorn M, Konno H, Matsumura T, Gohda J, Yoshida N, et al. Loss of Tifab, a del(5q) MDS gene, alters hematopoiesis through derepression of Toll-like receptor-TRAF6 signaling. J Exp Med. 2015; 212(11):1967-85.
9. Ganan-Gomez I, Wei Y, Starczynowski D T, Colla S, Yang H, Cabrero-Calvo M, et al. Deregulation of innate immune and inflammatory signaling in myelodysplastic syndromes. Leukemia. 2015; 29(7):1458-69.
10. Starczynowski D T, and Karsan A. Deregulation of innate immune signaling in myelodysplastic syndromes is associated with deletion of chromosome arm 5q. Cell Cycle. 2010; 9(5):855-6.
11. Zahid M F, Patnaik M M, Gangat N, Hashmi S K, and Rizzieri D A. Insight into the molecular pathophysiology of myelodysplastic syndromes: targets for novel therapy. Eur J Haematol. 2016; 97(4):313-20.
12. Li A J, and Calvi L M. The microenvironment in myelodysplastic syndromes: Niche-mediated disease initiation and progression. Exp Hematol. 2017; 55:3-18.
13. Trowbridge J J, and Starczynowski D T. Innate immune pathways and inflammation in hematopoietic aging, clonal hematopoiesis, and MDS. J Exp Med. 2021; 218(7).
14. Beachy S H, and Aplan P D. Mouse models of myelodysplastic syndromes. Hematol Oncol Clin North Am. 2010; 24(2):361-75.
15. Lin Y W, Slape C, Zhang Z, and Aplan P D. NUP98-HOXD13 transgenic mice develop a highly penetrant, severe myelodysplastic syndrome that progresses to acute leukemia. Blood. 2005; 106(1):287-95.
16. Sportoletti P, Grisendi S, Majid S M, Cheng K, Clohessy J G, Viale A, et al. Npml is a haploinsufficient suppressor of myeloid and lymphoid malignancies in the mouse. Blood. 2008; 111(7):3859-62.
17. Omidvar N, Kogan S, Beurlet S, le Pogam C, Janin A, West R, et al. BCL-2 and mutant NRAS interact physically and functionally in a mouse model of progressive myelodysplasia. Cancer Res. 2007; 67(24):11657-67.
18. Watanabe-Okochi N, Kitaura J, Ono R, Harada H, Harada Y, Komeno Y, et al. AML1 mutations induced MDS and MDS/AML in a mouse BMT model. Blood. 2008; 111(8):4297-308.
19. Wu M Y, Eldin K W, and Beaudet A L. Identification of chromatin remodeling genes Arid4a and Arid4b as leukemia suppressor genes. J Natl Cancer Inst. 2008; 100(17):1247-59.
20. Kato T, Watanabe N, Morishima Y, Fujita A, Ishizaki T, and Narumiya S. Localization of a mammalian homolog of diaphanous, mDia1, to the mitotic spindle in HeLa cells. J Cell Sci. 2001; 114(Pt 4):775-84.
21. Colucci-Guyon E, Niedergang F, Wallar B J, Peng J, Alberts A S, and Chavrier P. A role for mammalian diaphanous-related formins in complement receptor (CR3)-mediated phagocytosis in macrophages. Curr Biol. 2005; 15(22):2007-12.
22. Li F, and Higgs H N. Dissecting requirements for auto-inhibition of actin nucleation by the formin, mDia1. J Biol Chem. 2005; 280(8):6986-92.
23. Mei Y, Zhao B, Basiorka A A, Yang J, Cao L, Zhang J, et al. Age-related inflammatory bone marrow microenvironment induces ineffective erythropoiesis mimicking del(5q) MDS. Leukemia. 2018; 32(4):1023-33.
24. Lu L F, Boldin M P, Chaudhry A, Lin L L, Taganov K D, Hanada T, et al. Function of miR-146a in controlling Treg cell-mediated regulation of Th1 responses. Cell. 2010; 142(6):914-29.
25. Boldin M P, Taganov K D, Rao D S, Yang L, Zhao J L, Kalwani M, et al. miR-146a is a significant brake on autoimmunity, myeloproliferation, and cancer in mice. J Exp Med. 2011; 208(6):1189-201.
26. Yang L, Boldin M P, Yu Y, Liu C S, Ea C K, Ramakrishnan P, et al. miR-146a controls the resolution of T cell responses in mice. J Exp Med. 2012; 209(9):1655-70.
27. Starczynowski D T, Kuchenbauer F, Argiropoulos B, Sung S, Morin R, Muranyi A, et al. Identification of miR-145 and miR-146a as mediators of the 5q-syndrome phenotype. Nat Med. 2010; 16(1):49-58.
28. Eisenmann K M, Dykema K J, Matheson S F, Kent N F, DeWard A D, West R A, et al. 5q-myelodysplastic syndromes: chromosome 5q genes direct a tumor-suppression network sensing actin dynamics. Oncogene. 2009; 28(39):3429-41.
29. Peng J, Kitchen S M, West R A, Sigler R, Eisenmann K M, and Alberts A S. Myeloproliferative defects following targeting of the Drf1 gene encoding the mammalian diaphanous related formin mDia1. Cancer Res. 2007; 67(16):7565-71.
30. Votavova H, Urbanova Z, Kundrat D, Dostalova Merkerova M, Vostry M, Hruba M, et al. Modulation of the Immune Response by Deferasirox in Myelodysplastic Syndrome Patients. Pharmaceuticals (Basel). 2021; 14(1).
31. Shi X, Zheng Y, Xu L, Cao C, Dong B, and Chen X. The inflammatory cytokine profile of myelodysplastic syndromes: A meta-analysis. Medicine (Baltimore). 2019; 98(22):e15844.
32. Herold M, Schmalzl F, and Zwierzina H. Increased serum interleukin 6 levels in patients with myelodysplastic syndromes. Leuk Res. 1992; 16(6-7):585-8.
33. Pardanani A, Finke C, Lasho T L, Al-Kali A, Begna K H, Hanson C A, et al. IPSS-independent prognostic value of plasma CXCL10, IL-7 and IL-6 levels in myelodysplastic syndromes. Leukemia. 2012; 26(4):693-9.
34. Barreyro L, Chlon T M, and Starczynowski D T. Chronic immune response dysregulation in MDS pathogenesis. Blood. 2018; 132(15):1553-60.
35. Garbers C, Heink S, Korn T, and Rose-John S. Interleukin-6: designing specific therapeutics for a complex cytokine. Nat Rev Drug Discov. 2018; 17(6):395-412.
36. Tanaka T, Narazaki M, and Kishimoto T. IL-6 in inflammation, immunity, and disease. Cold Spring Harb Perspect Biol. 2014; 6(10):a016295.
37. Jones S A, and Jenkins B J. Recent insights into targeting the IL-6 cytokine family in inflammatory diseases and cancer. Nat Rev Immunol. 2018; 18(12):773-89.
38. Pellagatti A, Cazzola M, Giagounidis A, Perry J, Malcovati L, Della Porta M G, et al. Deregulated gene expression pathways in myelodysplastic syndrome hematopoietic stem cells. Leukemia. 2010; 24(4):756-64.
39. Rhyasen G W, Wunderlich M, Tohyama K, Garcia-Manero G, Mulloy J C, and Starczynowski D T. An MDS xenograft model utilizing a patient-derived cell line. Leukemia. 2014; 28(5):1142-5.
40. Wandt H, Haferlach T, Thiede C, and Ehninger G. WHO classification of myeloid neoplasms and leukemia. Blood. 2010; 115(3):748-9; author reply 9-50.
41. Wegrzyn J, Lam J C, and Karsan A. Mouse models of myelodysplastic syndromes. Leuk Res. 2011; 35(7):853-62.
42. Menssen A J, and Walter M J. Genetics of progression from MDS to secondary leukemia. Blood. 2020; 136(1):50-60.
43. Cahill C M, and Rogers J T. Interleukin (IL) 1beta induction of IL-6 is mediated by a novel phosphatidylinositol 3-kinase-dependent AKT/IkappaB kinase alpha pathway targeting activator protein-1. J Biol Chem. 2008; 283(38):25900-12.
44. Basiorka A A, McGraw K L, Eksioglu E A, Chen X, Johnson J, Zhang L, et al. The NLRP3 inflammasome functions as a driver of the myelodysplastic syndrome phenotype. Blood. 2016; 128(25):2960-75.
45. Burger R. Impact of interleukin-6 in hematological malignancies. Transfus Med Hemother. 2013; 40(5):336-43.
46. Yoshizaki K, Matsuda T, Nishimoto N, Kuritani T, Taeho L, Aozasa K, et al. Pathogenic significance of interleukin-6 (IL-6/BSF-2) in Castleman's disease. Blood. 1989; 74(4): 1360-7.
47. Bernad A, Kopf M, Kulbacki R, Weich N, Koehler G, and Gutierrez-Ramos J C. Interleukin-6 is required in vivo for the regulation of stem cells and committed progenitors of the hematopoietic system. Immunity. 1994; 1(9):725-31.
48. Voorzanger N, Touitou R, Garcia E, Delecluse H J, Rousset F, Joab I, et al. Interleukin (IL)-10 and IL-6 are produced in vivo by non-Hodgkin's lymphoma cells and act as cooperative growth factors. Cancer Res. 1996; 56(23):5499-505.
49. Kato H, Kinoshita T, Suzuki S, Nagasaka T, Murate T, Saito H, et al. Elevated serum interleukin-6 (IL-6) is derived from neoplastic lymphoid cells in patients with B-cell non-Hodgkin's lymphoma: correlation with extent of IL-6 expression and serum concentration. Br J Haematol. 1996; 92(4): 1014-21.
50. Garcia-Manero G, Gartenberg G, Steensma D P, Schipperus M R, Breems D A, de Paz R, et al. A phase 2, randomized, double-blind, multicenter study comparing siltuximab plus best supportive care (BSC) with placebo plus BSC in anemic patients with International Prognostic Scoring System low- or intermediate-1-risk myelodysplastic syndrome. Am J Hematol. 2014; 89(9):E156-62.
51. Rankin E B, Narla A, Park J K, Lin S, and Sakamoto K M. Biology of the bone marrow microenvironment and myelodysplastic syndromes. Mol Genet Metab. 2015; 116(1-2):24-8.
52. Mei Y, Han X, Liu Y, Yang J, Sumagin R, and Ji P. Diaphanous-related formin mDia2 regulates beta2 integrins to control hematopoietic stem and progenitor cell engraftment. Nat Commun. 2020; 11(1):3172.
53. Liu Y, Mei Y, Han X, Korobova F V, Prado M A, Yang J, et al. Membrane skeleton modulates erythroid proteome remodeling and organelle clearance. Blood. 2021; 137(3):398-409.
54. Mei Y, Zhao B, Basiorka A A, Yang J, Cao L, Zhang J, et al. Age-related inflammatory bone marrow microenvironment induces ineffective erythropoiesis mimicking del(5q) MDS. Leukemia. 2017.
55. Zhao B, Mei Y, Cao L, Zhang J, Sumagin R, Yang J, et al. Loss of pleckstrin-2 reverts lethality and vascular occlusions in JAK2V617F-positive myeloproliferative neoplasms. J Clin Invest. 2018; 128(1):125-40.
56. Chen J, Kao Y R, Sun D, Todorova T I, Reynolds D, Narayanagari S R, et al. Myelodysplastic syndrome progression to acute myeloid leukemia at the stem cell level. Nat Med. 2019; 25(1):103-10.

What is claimed:

1. A method of inhibiting disease progression in a subject with a high-risk myelodysplastic syndrome (MDS), the method comprising administering to the subject an inhibitor of the IL-6 signaling pathway in an amount effective to inhibit disease progression.

2. The method of claim 1, wherein the method inhibits the development of acute myeloid leukemia (AML) in the subject compared to an untreated control.

3. The method of claim 1, wherein the survival of the subject is extended compared to an untreated control.

4. The method of claim 1, wherein the method reduces the growth of blasts in the subject.

5. The method of claim 4, wherein the blasts are CD34+ cells.

6. The method of claim 4, wherein the blasts are CD34 negative cells that are morphologically defined as blasts.

7. The method of claim 1, wherein the inhibitor is an anti-IL-6 antibody or an anti-IL-6R antibody.

8. The method of claim 7, wherein the inhibitor is tocilizumab.

9. The method of claim 1, wherein the inhibitor is a recombinant gp130 Fc chimera protein.

10. The method of claim 1, wherein the subject is a human.

11. A method of treating low-blast count acute myeloid leukemia (AML) in a subject, the method comprising administering an inhibitor of the IL-6 signaling pathway in an amount effective to treat the AML.

12. The method of claim 11, wherein the inhibitor is an anti-IL-6 antibody or an anti-IL-6R antibody.

13. The method of claim 12, wherein the inhibitor is tocilizumab.

14. The method of claim 11, wherein the inhibitor is a recombinant gp130 Fc chimera protein.

15. The method of claim 11, wherein the subject is a human.

16. A method for identifying a therapeutic for preventing MDS to AML progression, the method comprising:
transplanting bone marrow cells from a mDia1/miR-146a double knockout (DKO) animal having MDS into a test animal;
administering a candidate therapeutic to the test animal; and
measuring the progression of MDS to AML in the test animal,
wherein a decrease in the progression of MDS to AML in the test animal compared to an untreated control animal transplanted with bone marrow cells from the DKO animal identifies the therapeutic.

17. The method of claim 16, wherein the progression of MDS to AML is measured by survival, wherein increased survival in the test animal compared to the control animal indicates a decrease in progression of MDS to AML.

18. The method of claim 16, wherein the progression of MDS to AML is measured by the growth of blasts, wherein reduced growth of blasts in the test animal compared to the control animal indicates a decrease in progress of MDS to AML.

19. The method of claim 16, wherein the progression of MDS to AML is measured by the development of pancytopenia, wherein reduced pancytopenia in the test animal compared to the control animal indicates a decrease in progress of MDS to AML.

20. The method of claim 16, wherein the test animal is a mouse.

21. The method of claim 16, further comprising lethally irradiating the test animal before the transplanting step.

22. The method of claim 16, wherein the DKO animal comprises increased levels of IL-6 receptor (IL-6R) in the bone marrow compared to a wild-type animal.
